(19) United States
(12) Patent Application Publication
(10) Pub. No.: US 2013/0059871 A1
(43) Pub. Date: Mar. 7, 2013

(54) PYRIMIDINE DERIVATIVES FOR THE TREATMENT OF AMYLOID-RELATED DISEASES

(71) Applicants: David Christopher HORWELL, Cambridge (GB); David Ian Carter SCOPES, Cambridge (GB)

(72) Inventors: David Christopher HORWELL, Cambridge (GB); David Ian Carter SCOPES, Cambridge (GB)

(21) Appl. No.: 13/459,739

(22) Filed: Apr. 30, 2012

Related U.S. Application Data
(63) Continuation of application No. 12/298,726, filed on Feb. 18, 2009, now abandoned.

(30) Foreign Application Priority Data
Apr. 27, 2007 (GB) 0608386.9

(51) Int. Cl. C07D 239/48; C07D 239/42; C07D 239/47; A61K 31/506; C07D 405/12; A61P 27/12; A61P 25/28; A61P 25/00; A61P 25/16; A61P 3/10; A61P 29/00; A61K 31/505; C07D 403/12

(52) U.S. Cl. 514/272; 544/332; 514/275; 544/330; 544/298

(57) ABSTRACT

The present invention provides (I) these compounds are useful in prevention and treatment of neurodegenerative disorders, such as Alzheimer's, Parkinson's and Huntington's as well as type II diabetes.

The present invention relates to novel heterocyclic compounds which are useful in the prevention and treatment of neurodegenerative disorders, such as Alzheimer's, Parkinson's and Huntington's as well as type II diabetes.

A number of incurable, ageing-related or degenerative diseases have been linked to a generic and fundamental pathogenic process of protein or peptide misfolding and aggregation called “amyloidosis”. These include Alzheimer's, Parkinson's and Huntington's diseases and type II diabetes. The amyloid deposits present in these diseases consist of particular peptides that are characteristic for each of these diseases but regardless of their sequence the amyloid fibrils have a characteristic β-sheet structure and share a common aggregation pathway. In each disease, a specific protein or peptide misfolds, adopts β-sheet structure and oligomerizes to form soluble aggregation intermediates en route to fibril formation ultimately forming insoluble amyloid fibres, plaques or inclusions. These insoluble forms of the aggregated protein or peptide form by the intermolecular association of β-strands into β-sheets. Recent evidence suggests that the soluble amyloid oligomers may be the principal cause of neurotoxicity.

The amyloidoses are defined as diseases in which normally soluble proteins accumulate in various tissues as insoluble deposits of fibrils that are rich in β-sheet structure and have characteristic dye-binding properties (Glenner, 1980a, 1980b). Although the specific polypeptides that comprise the deposits are different for each amyloidosis, the disorders have several key features in common. The most prominent of these is the ability of proteins that are highly soluble in biological fluids to be gradually converted into insoluble filamentous polymers enriched in β-pleated sheet conformation.

Furthermore, they tend to form by a similar molecular mechanism (by the intermolecular association of β-strands into extended β-sheets), so they tend to share a similar molecular structure and a common ability to bind certain dyes such as Congo Red and Thioflavin T (Selkoe 2003; Stefani 2004).

These diseases and disorders, which are collectively referred to herein as “amyloid-related diseases”, fall into two main categories: those which affect the brain and other parts of the central nervous system and those which affect other organs or tissues around the body, outside of the brain.

Examples of amyloid-related diseases which fall under these two categories are listed below in the following two sections, however many other examples of rare hereditary amyloid-related diseases are known which are not included here and more forms of amyloid-related disease are likely to be discovered in the future.

Neurodegenerative Diseases Associated with Amyloidosis

Many different neurodegenerative diseases are associated with the misfolding and aggregation of a specific protein or peptide in a particular part of the brain, or elsewhere in the central nervous system, depending on the specific disease (LeVine 2004; Caughey and Lansbury 2003; Dev et al. 2003; Taylor et al. 2002; Wood et al. 2003; Masino 2004; Ross and Poirier 2004; Soto and Castilla 2004; Forman et al. 2004). For example:

Various forms of Alzheimer's disease (AD/FAD) as well as Down's syndrome, hereditary cerebral hemorrhage with amyloidosis (HCHWA, Dutch type), cerebral amyloid angiopathy, and possibly also mild cognitive impairment and other forms of dementia are associated with the aggregation of a 40/42-residue peptide called β-amyloid, Aβ(1-40) or Aβ(1-42), which forms insoluble amyloid fibres and plaques in the cerebral cortex, hippocampus or elsewhere in the brain, depending on the specific disease;

Alzheimer's disease is also associated with the formation of neurofibrillary tangles by aggregation of a hyperphosphorylated protein called tau, which also occurs in frontotemporal dementia (Pick's disease);

Parkinson's disease (PD), dementia with Lewy bodies (DLB) and multiple system atrophy (MSA) are associated with the aggregation of a protein called α-synuclein, which results in the formation of insoluble inclusions called “Lewy bodies”;

Huntington's disease (HD), spinal and bulbar muscular atrophy (SBMA, also known as Kennedy's disease), dentatorubral pallidoluysian atrophy (DRPLA), different forms of spinocerebellar ataxia (SCA, types 1, 2, 3, 6 and 7), and possibly several other inheritable neurodegenerative diseases are associated with the aggregation of various proteins and peptides that contain abnormally expanded glutamine repeats (extended tracts of polyglutamine);

Creutzfeldt-Jakob disease (CJD), bovine spongiform encephalopathy (BSE) in cows, scrapie in sheep, kuru, Gerstmann-Straussler-Scheinker disease (GSS), fatal familial insomnia, and possibly all other forms of transmissible encephalopathy are associated with the self-propagating misfolding and aggregation of prion proteins;

Amyotrophic lateral sclerosis (ALS), and possibly also some other forms of motor neuron disease (MND) are associated with the aggregation of a protein called superoxide dismutase;

Familial British dementia (FBD) and familial Danish dementia (FDD) are respectively associated with aggregation of the ABri and ADan peptide sequences derived from the BRI protein; and

Hereditary cerebral hemorrhage with amyloidosis (HCHWA, Icelandic type) is associated with the aggregation of a protein called cystatin C.

Systemic Diseases Associated with Amyloidosis

In addition to the neurodegenerative diseases listed above, a wide variety of systemic ageing-related or degenerative diseases are associated with the misfolding and aggregation of a particular protein or peptide in various other tissues around the body, outside of the brain (Gejyo et al. 1985; Jaikaran and Clark 2001; Buxbaum 2004). For example:

Type II diabetes (also known as adult-onset diabetes, or non-insulin dependent diabetes mellitus) is associated with the aggregation of a 37-residue peptide called the islet amyloid polypeptide (IAPP, or “amylin”), which forms insoluble deposits that are associated with the progressive destruction of insulin-producing cells in the islets of Langerhans within the pancreas;

Dialysis-related amyloidosis (DRA) and prostatic amyloid are associated with the aggregation of a protein called β₂-microglobulin, either in bones, joints and tendons in DRA, which develops during prolonged periods of haemodialysis, or within the prostate in the case of prostatic amyloid;

Primary systemic amyloidosis, systemic AL amyloidosis and myeloma-associated amyloidosis are associated with the aggregation of immunoglobulin light chain (or in some cases immunoglobulin heavy chain) into insoluble amyloid deposits, which gradually accumulate in various major organs such as the liver, kidneys, heart and gastrointestinal (GI) tract;

Reactive systemic AA amyloidosis, secondary systemic amyloidosis, familial Mediterranean fever and chronic inflammatory disease are associated with the aggregation of serum amyloid A protein, which forms insoluble amyloid deposits that accumulate in major organs such as the liver, kidneys and spleen;

Senile systemic amyloidosis (SSA), familial amyloid polyneuropathy (FAP) and familial amyloid cardiomyopathy (FAC) are associated with the misfolding and aggregation of different mutants of transthyretin protein (TTR), which form insoluble inclusions in various organs and tissues such as the heart (especially in FAC), peripheral nerves (especially in FAP) and gastrointestinal (GI) tract;

Another form of familial amyloid polyneuropathy (FAP, type II) is associated with the aggregation of apolipoprotein AI in the peripheral nerves;

Familial visceral amyloidosis and hereditary non-neuropathic systemic amyloidosis are associated with misfolding and aggregation of various mutants of lysozyme, which form insoluble deposits in major organs such as the liver, kidneys and spleen;

Finnish hereditary systemic amyloidosis is associated with aggregation of a protein called gelsolin in the eyes (particularly in the cornea);

Fibrinogen α-chain amyloidosis is associated with aggregation of the fibrinogen A α-chain, which forms insoluble amyloid deposits in various organs such as the liver and kidneys;

Insulin-related amyloidosis occurs by the aggregation of insulin at the site of injection in diabetics;

Medullary carcinoma of the thyroid is associated with the aggregation of calcitonin in surrounding tissues;

Isolated atrial amyloidosis is associated with the aggregation of atrial natriuretic peptide (ANP) in the heart; and

Various forms of cataract are associated with the aggregation of y-crystallin proteins in the lens of the eyes.

### Pathogenic Mechanism of Amyloid-Related Diseases

While all these amyloid-related diseases share a common association with the pathogenic process of amyloidosis, the precise molecular mechanism by which this generic process of protein/peptide misfolding and aggregation is linked to the progressive degeneration of affected tissues is unclear. In some cases, including many of the systemic amyloid-related diseases, it is thought that the sheer mass of insoluble protein or peptide simply overwhelms the affected tissues, ultimately leading to acute organ failure. In other cases, including most of the neurodegenerative diseases listed above, however, the symptoms of disease develop with the appearance of only very small aggregates and it was suggested that these insoluble deposits are inherently toxic and might cause the progressive destruction of cells in some way, for example by causing inflammation and oxidative stress, or by directly interfering with cell membranes or other cellular components or processes.

More recently, however, it has been established that the specific proteins and peptides involved in at least some of these amyloid-related diseases form various soluble oligomeric species during their aggregation, which range in size from dimers and trimers, to much larger species comprising tens or even hundreds or thousands of protein or peptide monomers. Moreover, the oligomers are inherently toxic to cells in vitro in the absence of insoluble aggregates, and they appear to share a common structural feature as they can all be recognised by the same antibody despite the fact that they may be formed by proteins or peptides with very different amino acid sequences (Kayed et al. 2003; Glabe 2004; Walsh et al. 2002; Walsh and Selkoe 2004).

The molecular structure of these toxic soluble oligomers is not known and the precise mechanism by which they kill cells is also unclear, but several theories have been proposed. According to just one theory called the “channel hypothesis”, for example, the oligomers form heterogeneous pores or leaky ion channels, which allow ions to flow freely through cell membranes, thereby destroying their integrity which ultimately causes cell death (Kagan et al. 2002). Alternatively, or in addition, the oligomers may form protofibrils which kill cells by a similar or completely different mechanism.

Regardless of the precise pathogenic mechanism, however, an overwhelming amount of evidence has now been accumulated which suggests that the general process of protein/peptide aggregation is the primary cause of all these, and possibly other, different amyloid-related diseases.

The present invention relates to chemical compounds and compositions which are inhibitors of amyloid toxicity and as such have use in the treatment of amyloid-related diseases and disorders.

WO03045923, in the name of Sankyo, describes a limited class of bis-anilino heterocycles which have been shown to inhibit amyloid toxicity. We have found unexpectedly that the aniline linkage can be replaced by a number of alternatives which provide molecules which are distinct from the aforementioned compounds in their activity profile. In addition, we have found that it is not necessary for both of the substituents (X and Y in formula I) on the central heterocyclic core ring to be attached adjacent to the ring nitrogen atoms of said heterocycle.

Thus, in a first aspect, the present invention provides a compound of formula (I) or a pharmaceutically acceptable salt or prodrug thereof:

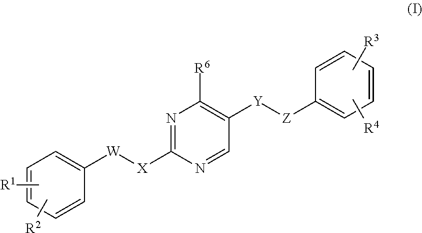

wherein
X and Y are independently NR⁵ or O;
W and Z are independently a bond or (CH₂)ₘCH(R⁷)(CH₂)ₙ;
m=0-1 and n=0-2;
R¹ and R² are independently hydrogen, halogen, CF₃, OR⁸, OR⁹, NR⁹R¹⁰, NR⁹COR¹¹, NR⁹SO₂R¹¹, SO₂NR⁹R¹⁰, SO₂R¹¹ or C₁₋₆ alkyl optionally and independently substituted by one or more of hydroxyl, C₁₋₆ alkoxy, halogen or NR⁹R¹⁰;
R³ is hydrogen, halogen, CF₃, OR⁸, COOR⁹, CONR⁹R¹⁰ or SO₂R¹¹;
R⁴ is hydrogen, halogen, CF₃, OR⁹, NR⁹R¹⁰, NR⁹COR¹¹, NR⁹SO₂R¹¹, SO₂NR⁹R¹⁰, or C₁₋₆ alkyl optionally substituted by hydroxyl, C₁₋₆ alkoxy or NR⁹R¹⁰;
or when R³ and R⁴ are positioned ortho and taken together form —O(CH₂)ₙO—, where n is 1-3;
R⁵ is hydrogen or C₁₋₆ alkyl optionally substituted by hydroxyl, C₁₋₆ alkoxy or NR⁹R¹⁰;
R⁶ is hydrogen, C₁₋₆ alkyl, C₁₋₆ alkoxy or NR⁹R¹⁰;
R⁷ is hydrogen, C₁₋₆ alkyl, phenyl or C₁₋₃ alkylphenyl wherein said phenyl groups are optionally substituted by one or more substituents selected from halogen, C₁₋₆ alkyl, CF₃, OCF₃ or OR⁹;
R⁸ is hydrogen or C₁₋₆ alkyl optionally substituted by OR⁹ or NR⁹R¹⁰;
R⁹ is hydrogen, C₁₋₆ alkyl or C₁₋₃ alkylphenyl wherein said phenyl group is optionally substituted by one or more substituents selected from halogen, C₁₋₆ alkyl, CF₃, OR⁸, NR⁹R¹⁰ or OCF₃;
R¹⁰ is hydrogen, C₁₋₆ alkyl, C₁₋₆ alkenyl, phenyl or C₁₋₃ alkylphenyl wherein said phenyl groups are optionally substituted by one or more substituents selected from halogen, C₁₋₆ alkyl, CF₃, OR⁸ or OCF₃;
or the groups R⁹ and R¹⁰ when they are attached to a nitrogen atom may together form a 5- or 6-membered ring which optionally contains one further heteroatom selected from NR⁹, S and O; and
R¹¹ is C₁₋₆ alkyl or a phenyl group optionally substituted by one or more substituents selected from halogen, C₁₋₆ alkyl, CF₃, OCF₃ or OR⁸.

Preferably R¹ and R² are independently CHOHCF₃.

As used herein “positioned ortho” means that R₃ and R₄ are on adjacent carbon atoms. They can be taken together to form —O(CH₂)ₙO—, where n is 1-3. n is preferably 1, 2, or 3. Examples of such groups include —OCH₂O—, —OCH₂CH₂O— or —OCH₂CH₂CH₂O—. These groups together with the carbon atoms to which they are attached form a 5-, 6- or 7-membered ring.

In a preferred embodiment the invention provides a compound of formula (I) or a pharmaceutically acceptable salt or prodrug thereof:

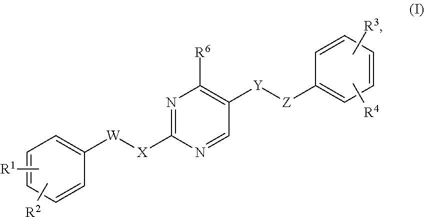

wherein
X and Y are independently NR⁵ or O;
W and Z are independently a bond or (CH₂)ₘCH(R⁷)(CH₂)ₙ;
m=0-1, n=0-2;
R¹ and R² are independently hydrogen, halogen, CF₃, OR⁸, NR⁹R¹⁰, NR⁹COR¹¹, NR⁹SO₂R¹¹ or C₁₋₆ alkyl optionally substituted by hydroxyl, C₁₋₆ alkoxy or NR⁹R¹⁰;
R³ is hydrogen, halogen, CF₃, OR⁸, COOR⁹, CONR⁹R¹⁰ or SO₂R¹¹;
R⁴ is hydrogen, halogen, CF₃, OR⁹, NR⁹R¹⁰, NR⁹COR¹¹, NR⁹SO₂R¹¹ or C₁₋₆ alkyl optionally substituted by hydroxyl, C₁₋₆ alkoxy or NR⁹R¹⁰;
R⁵ is hydrogen or C₁₋₆ alkyl optionally substituted by hydroxyl, C₁₋₆ alkoxy or NR⁹R¹⁰;
R⁶ is hydrogen, C₁₋₆ alkyl, C₁₋₆ alkoxy or NR⁹R¹⁰;
R⁷ is hydrogen, C₁₋₆ alkyl, phenyl or C₁₋₃ alkylphenyl wherein said phenyl groups are optionally substituted by one or more substituents selected from halogen, C₁₋₆ alkyl, CF₃, OCF₃ or OR⁹;
R⁸ is hydrogen or C₁₋₆ alkyl optionally substituted by NR⁹R¹⁰;
R⁹ is hydrogen, C₁₋₆ alkyl or C₁₋₃ alkylphenyl wherein said phenyl group is optionally substituted by one or more substituents selected from halogen, C₁₋₆ alkyl, CF₃, OR⁸, NR⁹R¹⁰ or OCF₃;
R¹⁰ is hydrogen, C₁₋₆ alkyl, C₁₋₆ alkenyl, phenyl or C₁₋₃ alkylphenyl wherein said phenyl groups are optionally substituted by one or more substituents selected from halogen, C₁₋₆ alkyl, CF₃, OR⁸ or OCF₃;
or the groups R⁹ and R¹⁰ when they are attached to a nitrogen atom may together form a 5- or 6-membered ring which optionally contains one further heteroatom selected from NR⁹, S and O; and
R¹¹ is C₁₋₆ alkyl or a phenyl group optionally substituted by one or more substituents selected from halogen, C₁₋₆ alkyl, CF₃, OCF₃ or OR⁸.

Preferably

R¹ and R² are independently hydrogen, halogen, CF₃, OR⁸ or NR⁹R¹⁰;
R³ is hydrogen, F, or OR⁸;
R⁴ is hydrogen, halogen, CF₃, OR⁹ or NR⁹R¹⁰;
R⁵ is hydrogen or C₁₋₆ alkyl optionally substituted by hydroxyl, C₁₋₆ alkoxy or NR⁹R¹⁰;
R⁶ is hydrogen, C₁₋₆ alkyl, C₁₋₆ alkoxy or NR⁹R¹⁰;
R⁷ is hydrogen, C₁₋₆ alkyl;
R⁸ is hydrogen or C₁₋₆ alkyl optionally substituted by OR⁹ or NR⁹R¹⁰;
R⁹ is hydrogen, C₁₋₆ alkyl or C₁₋₃ alkylphenyl wherein said phenyl groups are optionally substituted by one or more substituents selected from halogen, C₁₋₆ alkyl, CF₃, OR⁸, NR⁹R¹⁰ or OCF₃;
R¹⁰ is hydrogen, C₁₋₆ alkyl, C₁₋₆ alkenyl, phenyl or C₁₋₃ alkylphenyl wherein said phenyl groups are optionally substituted by one or more substituents selected from halogen, C₁₋₆ alkyl, CF₃, OR⁸ or OCF₃;
or the groups R⁹ and R¹⁰ when they are attached to a nitrogen atom may together form a 5- or 6-membered ring which optionally contains one further heteroatom selected from NR⁹, S and O; and
R¹¹ is C₁₋₆ alkyl or a phenyl group optionally substituted by one or more substituents selected from halogen, C₁₋₆ allyl, CF₃, OCF₃ or OR⁸.
m=0 and n=0-1

Preferred compounds are
2,5-Bis-(3-hydroxyphenylamino)pyrimidine
2-(3-Hydroxyphenylamino)-5-[phenyl(methyl)amino]pyrimidine
2-(3-Hydroxyphenylamino)-5-(4-fluorophenoxy)pyrimidine
2-(3-Hydroxyphenylamino)-5-[4-fluorophenyl(methyl)amino]pyrimidine
5-[4-Fluorophenyl(methyl)amino]2-(phenylamino)-pyrimidine
2-(3-Fluorophenylamino)5-[4-fluorophenyl(methyl)amino]pyrimidine
2-(3-Hydroxyphenylamino)-5-(3-methoxyphenoxy)pyrimidine
2-(3-Hydroxyphenylamino)-5-(3-dimethylaminophenoxy)pyrimidine
2-(3-Hydroxyphenylamino)-5-(2,3-dihydrobenzo[1,4]dioxin-6-yl)oxypyrimidine
2-(3-Hydroxyphenylamino)-5-[3-(pyrrolidin-1-yl)phenoxy]pyrimidine
5-(4-Fluorophenoxy)-2-(phenylamino)pyrimidine
5-(3-Dimethylaminophenoxy)-2-(phenylamino)pyrimidine
5-(2,3-Dihydrobenzo[1,4]dioxin-6-yl)oxy-2-(phenylamino)pyrimidine
5-(4-Fluorophenoxy)-2-(3-methoxyphenylamino)pyrimidine
5-(3-Dimethylaminophenoxy)-2-(3-methoxyphenylamino)primidine
5-(2,3-Dihydrobenzo[1,4]dioxin-6-yl)oxy-2-(3-methoxyphenylamino)pyrimidine
5-(4-Fluorophenoxy)-2-(3-fluorophenylamino)pyrimidine
5-(3-Dimethylaminophenoxy)-2-(3-fluorophenylamino)pyrimidine
5-(2,3-Dihydrobenzo[1,4]dioxin-6-yl)oxy-2-(3-fluorophenylamino)pyrimidine

The term “alkyl” as used herein whether on its own or as part of a larger group e.g. “alkoxy” or “alkylphenyl” includes both straight and branched chain radicals, including but not limited to methyl, ethyl, n-propyl, isopropyl, n-butyl, sec-butyl and tert-butyl. The term alkyl also includes those radicals wherein one or more hydrogen atoms are replaced by fluorine, e.g. CF₃.

The term “alkenyl” and “alkynyl” as used herein includes both straight and branched chain radicals.

The term “halogen” as used herein includes fluorine, chlorine and bromine

The compounds of the first aspect may be provided as a salt, preferably as a pharmaceutically acceptable salt of compounds of formula (I). Examples of pharmaceutically acceptable salts of these compounds include those derived from organic acids such as acetic acid, malic acid, tartaric acid, citric acid, lactic acid, oxalic acid, succinic acid, fumaric acid, maleic acid, benzoic acid, salicylic acid, phenylacetic acid, mandelic acid, methanesulphonic acid, benzenesulphonic acid and p-toluenesulphonic acid, mineral acids such as hydrochloric and sulphuric acid and the like, giving methanesulphonate, benzenesulphonate, p-toluenesulphonate, hydrochloride and sulphate, and the like, respectively or those derived from bases such as organic and inorganic bases. Examples of suitable inorganic bases for the formation of salts of compounds for this invention include the hydroxides, carbonates, and bicarbonates of ammonia, lithium, sodium, calcium, potassium, aluminium, iron, magnesium, zinc and the like. Salts can also be formed with suitable organic bases. Such bases suitable for the formation of pharmaceutically acceptable base addition salts with compounds of the present invention include organic bases, which are nontoxic and strong enough to form salts. Such organic bases are already well known in the art and may include amino acids such as arginine and lysine, mono-, di-, or trihydroxyalkylamines such as mono-, di-, and triethanolamine, choline, mono-, di-, and trialkylamines, such as methylamine, dimethylamine, and trimethylamine, guanidine; N-methylglucosamine; N-methylpiperazine; morpholine; ethylenediamine; N-benzylphenethylamine; tris(hydroxymethyl)aminomethane; and the like.

Salts may be prepared in a conventional manner using methods well known in the art. Acid addition salts of said basic compounds may be prepared by dissolving the free base compounds according to the first aspect of the invention in aqueous or aqueous alcohol solution or other suitable solvents containing the required acid. Where a compound of the invention contains an acidic function, a base salt of said compound may be prepared by reacting said compound with a suitable base. The acid or base salt may separate directly or can be obtained by concentrating the solution e.g. by evaporation.

The pharmaceutically acceptable prodrugs of the compounds of formula (I) may be prepared by methods well known to those skilled in the art. A prodrug is commonly described as an inactive or protected derivative of an active ingredient or a drug, which is converted to the active ingredient or drug in the body. Examples of prodrugs include pharmaceutically acceptable esters, including C₁-C₆ alkyl esters and pharmaceutically acceptable amides, including secondary C₁-C₃ amides.

The compounds of the invention may exist in the form of optical isomers, e.g. diastereoisomers and mixtures of isomers in all ratios, e.g. racemic mixtures. The invention includes in particular the isomeric forms (R or S). The different isomeric forms may be separated or resolved one from the other by conventional methods, or any given isomer may be obtained by conventional synthetic methods or by stereospecific or asymmetric synthesis. Where a compound contains an alkene moiety, the alkene can be presented as a cis or trans isomer or a mixture thereof. When an isomeric form of a compound of the invention is provided substantially free of other isomers, it will preferably contain less than 5% w/w, more preferably less than 2% w/w and especially less than 1% w/w of the other isomers.

Since the compounds of the invention are intended for use in pharmaceutical compositions, it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5%, e.g. 10 to 59% of a compound of the formula (I).

A compound of formula (I), wherein R¹, R², R³, R⁴, R⁶ are as defined for formula (I), X═Y═NR⁵ and Z═W=bond, may be prepared from a compound of formula (II)

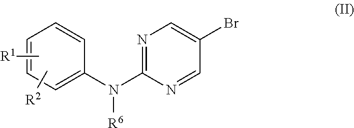

wherein R¹, R² and R⁵ are as defined in formula (I) by treatment with an appropriate aniline in the presence of a suitable catalyst such as tris(dibenzylideneacetone)-palladium(0), a phosphine ligand such as 4,5-bis(diphenylphosphino)-9,9-dimethylxanthene and a base such as cesium carbonate in a solvent such as 1,4-dioxan with heating.

A compound of formula (II) wherein R¹, R² and R⁵ are as defined in formula (I), may be prepared by treatment of 2-chloro-5-bromopyrimidine with one equivalent of an appropriate aniline in a suitable solvent such as an alcohol and heating in a sealed tube under microwave irradiation.

A compound of formula (I), wherein R¹, R², R³, R⁴, R⁶ are as defined for formula (I), X═O, Y═NR⁵ and Z═W=bond, may be prepared from a compound of formula (III)

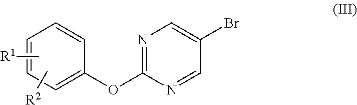

wherein R¹, R² are as defined in formula (I) by treatment with an appropriate aniline in the presence of a suitable catalyst such as tris(dibenzylideneacetone)-palladium(0), a phosphine ligand such as 4,5-bis(diphenylphosphino)-9,9-dimethylxanthene and a base such as cesium carbonate in a solvent such as 1,4-dioxan with heating.

A compound of formula (III) wherein R¹, R² are as defined in formula (I), may be prepared by treatment of 2-chloro-5-bromopyrimidine with one equivalent of an appropriate phenol in the presence of a suitable base such as cesium carbonate in a suitable solvent such DMF and applying heat.

A compound of formula (I), wherein R¹, R², R³, R⁴, R⁶ are as defined for formula (I), X═NR⁵, Y═O and Z═W=bond, may be prepared from a compound of formula (II) wherein R¹, R² and R⁶ are as defined in formula (I) by treatment with bis(pinacolato)diborane, [1,1′-bis(diphenylphosphino)ferrocene]dichloropalladium (II) in the presence of a base in a suitable solvent such as DMSO with heating. The resultant boronic ester is treated with aqueous hydrogen peroxide in a suitable co-solvent, such as methanol, and the resultant hydroxyl compound coupled via an arylboronic acid using a copper catalyst such as copper (II) acetate in the presence of triethylamine and powdered 4 Å molecular sieves in a suitable solvent such as dichloromethane, at room temperature or with application of heat.

A compound of formula (I), wherein R¹, R², R³, R⁴, R⁶ are as defined for formula (I), X═NR⁵ or O, W is (CH₂)ₘCH(R⁷)(CH₂)ₙ, Y═NR⁵ and Z=bond, may be prepared from a compound of formula (IV), wherein R¹, R², R⁷, X, m and n are as defined in formula (I), by treatment either with an appropriate aniline in the presence of a suitable catalyst such as tris(dibenzylideneacetone)-palladium(0) a phosphine ligand such as 4,5-bis(diphenylphosphino)-9,9-dimethylxanthene and a base such as cesium carbonate in a solvent such as 1,4-dioxan with heating.

A compound of formula (I), wherein R¹, R², R³_{,} R⁴, R⁶ are as defined for formula (I), X═NR⁵ or O, W is (CH₂)ₘCH(R⁷)(CH₂)ₙ, Y═O and Z=bond, may be prepared from a compound of formula (IV), wherein R¹, R², R⁷, X, m and n are as defined in formula (I), by treatment with bis(pinacolato)diborane, [1,1′-bis(diphenylphosphino)-ferrocene]dichloropalladium (II) in the presence of a base in a suitable solvent such as DMSO with heating. The resultant boronic ester is treated with aqueous hydrogen peroxide in a suitable co-solvent, such as methanol, and the resultant hydroxyl compound coupled via an arylboronic acid using a copper catalyst such as copper (II) acetate in the presence of triethylamine and powdered 4 Å molecular sieves in a suitable solvent such as dichloromethane, at room temperature or with application of heat.

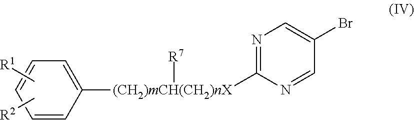

A compound of formula (IV) wherein R¹, R², R⁷, X, m and n are as defined in formula (I), may be prepared by treatment of 2-chloro-5-bromopyrimidine with one equivalent of the appropriate amine (V) or alcohol (VI) in a suitable solvent such as DMF, optionally in the presence of a base such as sodium hydride, and applying heat.

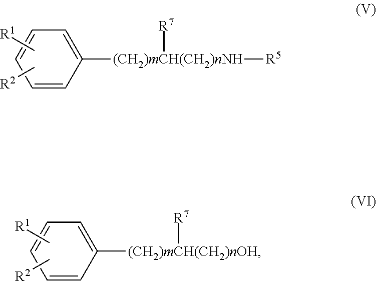

A compound of formula (I), wherein R¹, R², R³, R⁴, R⁶ are as defined for formula (I), X═NR⁵ or O, W is a bond, Y═NR⁵ and Z═(CH₂)ₘCH(R⁷)(CH₂)ₙ may be prepared from (VII) wherein R¹, R² and X are as defined in formula (I), by treatment with either a compound of formula (VIII), when m is =1 in formula (I), or a compound of formula (IX) when m=0 in formula (I), under reductive amination conditions, for example using sodium cyanoborohydride in a protic solvent such as methanol at a mildly acidic pH for example 4-5.

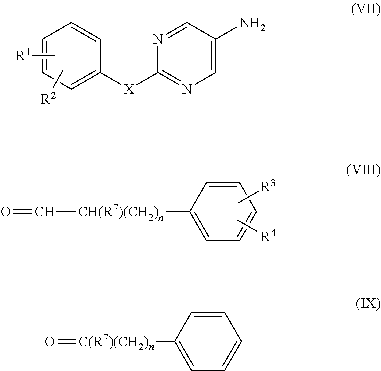

A compound of formula (VII) wherein R¹, R² are as defined for formula (I) and X═NR⁵ may be prepared by treatment of 2-chloro-5-nitropyrimidine with one equivalent of an appropriate aniline in a suitable solvent such as an alcohol and heating in a sealed tube under microwave irradiation. To complete the preparation of compounds of formula (VII) the nitro group can be reduced by standard methods.

A compound of formula (VII) wherein R¹, R² are as defined for formula (I) and X═O may be prepared by treatment of 2-chloro-5-nitropyrimidine with one equivalent of an appropriate phenol in the presence of a suitable base such as cesium carbonate in a suitable solvent such DMF and applying heat. To complete the preparation of compounds of formula (VII) the nitro group can be reduced by standard methods.

It will be appreciated by someone skilled in the art that by using the methods described above in various combinations it will be possible to synthesise other derivatives encompassed in the general formula (I).

It will also be appreciated that the aniline, phenol, amine, alcohol, aldehyde and ketone building blocks used in the synthesis of compounds of general formula (I) are either commercially available or can be synthesised by methods known in the art.

During the synthesis of the compounds of formula (I), including those of formula (IA) described below, labile functional groups in the intermediate compounds, e.g. hydroxyl, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in for example Protective Groups in Organic Chemistry, T. W. Greene and P. G. M. Wuts (Wiley-Interscience, New York, 2^{nd} edition, 1991).

In another aspect the invention provides an intermediate in the synthesis of a compound of Formula (I) of formula (IA)

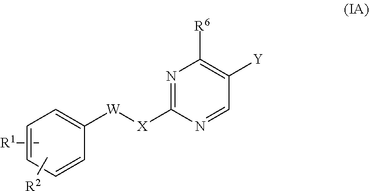

wherein R₁, R₂, W, X and R₆ are as defined above and Y is Cl, Br, I or OH. Y is preferably Br.

Preferred intermediates are selected from
2-(3-Trifluoromethylphenylamino)-5-bromopyrimidine;
2-(3,4-Dichlorophenylamino)-5-bromopyrimidine;
2-(3-Benzyloxyphenyl-N-tert-butyloxycarbonylamino)-5-bromopyrimidine;
2-(3-Benzyloxyphenyl-N-tert-butyloxycarbonylamino)-5-hydroxypyrimidine;
5-Bromo-2-(N-tert-butyloxycarbonylphenylamino)pyrimidine;
5-Bromo-2-(phenylamino)pyrimidine;
2-(Phenylamino)-5-(4,4,5,5-tetramethyl-[1,3,2]dioxaborolan-2-yl)pyrimidine;
5-Hydroxy-2-(phenylamino)pyrimidine;
1-[4-(5-Bromopyrimidin-2-ylamino)phenyl]-2,2,2-trifluoroethanone; or
1-[4-(5-Bromopyrimidin-2-ylamino)phenyl]-2,2,2-trifluoroethanol.

The pharmaceutically effective compounds of formula (I) may be administered in conventional dosage forms prepared by combining a compound of formula (I) (“active ingredient”) with standard pharmaceutical carriers or excipients according to conventional procedures well known in the art. The procedures may involve mixing, granulating and compressing or dissolving the ingredients as appropriate to the desired preparation.

Thus, in a third aspect, the present invention provides a pharmaceutical composition comprising a compound of formula (I), or a pharmaceutically acceptable salt or prodrug thereof, together with one or more pharmaceutically acceptable carriers or excipients.

The active ingredient or pharmaceutical composition can be administered simultaneously, separately or sequentially with another appropriate treatment for the amyloid-related disease being treated.

The active ingredient or pharmaceutical composition may be administered to a subject by any of the routes conventionally used for drug administration, for example they may be adapted for oral (including buccal, sublingual), topical (including transdermal), nasal (including inhalation), rectal, vaginal or parenteral (including subcutaneous, intramuscular, intravenous or intradermal) administration to mammals including humans. The most suitable route for administration in any given case will depend upon the particular compound or pharmaceutical composition, the subject, and the nature and composition and severity of the disease and the physical condition of the subject. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Tablets and capsules for oral administration may be in unit dose presentation form, and may contain conventional excipients such as binding agents, for example syrup, acacia, gelatin, sorbitol, tragacanth, or polyvinylpyrrolidine; filler, for example lactose, sugar, maize-starch, calcium phosphate, sorbitol or glycine; tabletting lubricants; for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Pharmaceutical compositions adapted for topical administration may be formulated as ointments, creams, suspensions, lotions powders, solutions, pastes, gels, sprays, aerosols or oils and may contain appropriate conventional additives such as preservatives, solvents to assist drug penetration and emollients in ointments and creams. Such applications include those to the eye or other external tissues, for example the mouth and skin and the compositions are preferably applied as a topical ointment or cream. When formulated in an ointment, the active ingredient may be employed with either a paraffinic or a water miscible ointment base. Alternatively, the active ingredient may be formulated in a cream with an oil-in-water cream base or a water-in-oil base. The composition may also contain compatible conventional carriers, such as cream or ointment bases and ethanol or oleyl alcohol for lotions.

Pharmaceutical compositions adapted for topical administration to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for transdermal administration may be presented as discrete patches intended to remain in intimate contact with the epiderma of the recipient for a prolonged period of time. For example, the active ingredient may be delivered from the patch by iontophoresis as generally described in Pharmaceutical Research, 3(6), 318 (1986).

Pharmaceutical compositions adapted for controlled or sustained release may be administered by injection, for example by the subcutaneous route.

Pharmaceutical compositions adapted for nasal administration wherein the carrier is a solid include coarse powder having a particle size for example in the range of 20-500 microns which is administered by rapid inhalation through the nasal passage from a container of the powder held close to the nose. Suitable compositions wherein the carrier is a liquid, for administration as a nasal spray or as nasal drops, include aqueous or oil solutions of an active ingredient.

Pharmaceutical compositions adapted for administration by inhalation include fine particle dusts or mists which may be generated by means of various types of metered dose pressurise aerosols, nebulizers or insufflators.

Pharmaceutical compositions adapted for rectal administration may be presented as suppositories or enemas. Suppositories will contain conventional suppository bases, e.g. cocoa-butter or other glyceride.

Pharmaceutical compositions adapted for vaginal administration may be presented as pessaries, tampons, creams, gels, pastes, foams or spray compositions.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solution and suspensions may be prepared from sterile powders, granules and tablets.

For parenteral administration, fluid unit dosage forms are prepared utilising the active ingredient and a sterile vehicle, water being preferred. The active ingredient, depending on the vehicle and concentration used, can be either suspended or dissolved in the vehicle. In preparing solutions the active ingredient can be dissolved in water for injection and filter sterilised before filling into a suitable vial or ampoule and sealing.

Advantageously, agents such as local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the active ingredient is suspended in the vehicle instead of being dissolved and sterilisation cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

The pharmaceutical compositions according to the invention are preferably adapted for oral administration.

It should be understood that in addition to the ingredients particularly mentioned above, the compositions may also include other agents conventional in the art having regard to the type of formulation in question, for example those suitable for oral administration may include flavouring agents. They may also contain therapeutically active agents in addition to the compounds of the present invention. Such carriers may be present as from about 1% up to about 98% of the formulation. More usually they will form up to about 80% of the formulation.

The compositions may contain from 0.1% by weight, preferably from 10-60% by weight, of the active material, depending on the method of administration.

Pharmaceutical compositions may be presented in unit dose forms containing a predetermined amount of active ingredient per dose. Such a unit may contain for example 0.1 mg/kg to 750 mg/kg, more preferably 0.1 mg/kg to 10 mg/kg depending on the condition being treated, the route of administration and the age, weight and condition of the patient. Preferred unit dosage compositions are those containing a daily dose or sub-dose, as herein above recited, or an appropriate fraction thereof, of an active ingredient.

It will be recognised by one of skill in the art that the optimal quantity and spacing of individual dosages of compounds in the first and second aspects of the invention will be determined by the nature and extent of the condition being treated the form, route and site of administration, and the particular subject being treated, and that such optimums can be determined by conventional techniques. It will also be appreciated by one of skill in the art that the optimal course of treatment, i.e., the number of doses of the aforementioned compounds given per day for a defined number of days, can be ascertained by those skilled in the art using conventional course of treatment determination tests.

Depending on the route of administration, the chemical compound or composition may be required to be coated in a material to protect it from the action of enzymes, acids and other natural conditions which may inactivate it.

In order to administer the chemical compound or composition by other than parenteral administration, it may be coated by, or administered with, a material to prevent its inactivation. For example, it may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether.

Liposomes include water-in-oil-in-water CGF emulsions as well as conventional liposomes.

The active chemical compound or composition may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical compositions or formulations suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active chemical compound or composition in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the chemical compound or composition is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. The amount of active compound in such therapeutically useful compositions is such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In other aspects, the present invention provides:

1. The use of a compound of the invention in the manufacture of a medicament for the treatment of an amyloid-related disease. In particular, the medicament is for the treatment of;
a) any form of Alzheimer's disease (AD or FAD);
b) any form of mild cognitive impairment (MCI) or senile dementia;
c) Down's syndrome;
d) cerebral amyloid angiopathy, inclusion body myositis, hereditary cerebral hemorrhage with amyloidosis (HCHWA, Dutch type), or age-related macular degeneration (ARMD);
e) fronto-temporal dementia;
f) any form of Parkinson's disease (PD) or dementia with Lewy bodies;
g) Huntington's disease (HD), dentatorubral pallidoluysian atrophy (DRPLA), spinocerebellar ataxia (SCA, types 1, 2, 3, 6 and 7), spinal and bulbar muscular atrophy (SBMA, Kennedy's disease), or any other polyglutamine disease;
h) Creutzfeldt-Jakob disease (CJD), bovine spongiform encephalopathy (BSE) in cows, scrapie in sheep, kuru, Gerstmann-Straussler-Scheinker disease (GSS), fatal familial insomnia, or any other transmissible encephalopathy that is associated with the aggregation of prion proteins;
i) amyotrophic lateral sclerosis (ALS) or any other form of motor neuron disease;
j) familial British dementia (FBD) or familial Danish dementia (FDD);
k) hereditary cerebral hemorrhage with amyloidosis (HCHWA, Icelandic type);
l) type U diabetes (adult onset diabetes, or non-insulin dependent diabetes mellitus, NIDDM);
m) dialysis-related amyloidosis (DRA) or prostatic amyloid;
n) primary systemic amyloidosis, systemic AL amyloidosis, or nodular AL amyloidosis;
o) myeloma associated amyloidosis;
p) systemic (reactive) AA amyloidosis, secondary systemic amyloidosis, chronic inflammatory disease, or familial Mediterranean fever;
q) senile systemic amyloidosis, familial amyloid polyneuropathy, or familial cardiac amyloid;
r) familial visceral amyloidosis, hereditary non-neuropathic systemic amyloidosis, or any other lysozyme-related amyloidosis;
s) Finnish hereditary systemic amyloidosis;
t) fibrinogen α-chain amyloidosis;
u) insulin-related amyloidosis;
v) medullary carcinoma of the thyroid;
w) isolated atrial amyloidosis;
x) any form of cataract; and
y) any other amyloid-related disease that is associated with the misfolding or aggregation of a specific target amyloid-forming protein or peptide into toxic soluble oligomers, protofibrils, ion channels, insoluble amyloid fibres, plaques or inclusions.
2. A method for the treatment of an amyloid-related disease, which comprises the step of administering to a subject an effective amount of a compound or pharmaceutical composition of the invention.

### EXAMPLES

The following examples are to be construed merely illustrative and not a limitation on the scope of the invention in any way.

### General

All reagents and solvents were commercial grade and were used as received without further purification. Petroleum ether refers to the fraction boiling between 40 and 60° C. Column chromatography was performed on Matrex® silica gel 60 (35-70 micron). ¹H NMR spectra were recorded on a Bruker DPX400 at 400 MHz. Chemical shifts for ¹H NMR spectra are given in parts per million and either tetramethylsilane (0.00 ppm) or residual solvent peaks were used as internal reference. Splitting patterns are designated as follows: s, singlet; d, doublet; t, triplet; in, multiplet; br, broad. LCMS analyses were performed using a Micromass ZQ or Platform LC instrument with atmospheric pressure chemical ionisation (APCI) or electrospray ionisation (ESI) on a Waters Xterra MS reverse-phase column (5μ C18, 100×4.6 mm) eluting at 2 ml/min with a gradient of acetonitrile/water containing 7 mM ammonia. Purity was assessed as the integral over the window 210-400 nm (Waters or HP DAD).

### Example 1

### 2,5-Bis-(3-benzyloxyphenylamino)pyrimidine

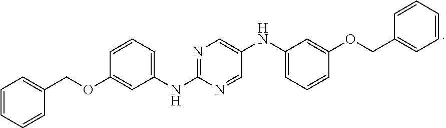 [SMILES: c1ccc(COc2cccc(Nc3cnc(Nc4cccc(OCc5ccccc5)c4)nc3)c2)cc1]

A suspension of 2-chloro-5-bromopyrimidine (300 mg, 1.55 mol), 3-benzyloxyaniline (680 mg, 3.41 mmol), tris(dibenzylideneacetone)palladium(0) (56 mg, 61.1 μmol), 4,5-bis(diphenylphosphino)-9,9-dimethylxanthene (71 mg, 122.7 μmol) and cesium carbonate (1.21 g, 3.72 mmol) in degassed 1,4-dioxan (7 mL) was heated at 80° C. for 4 days. After cooling to room temperature, the mixture was diluted with ethyl acetate and washed with water, 0.5 M hydrochloric acid and brine. The organic phase was dried (MgSO₄) and the solvent removed under reduced pressure to give a thick orange-brown oil. The crude product was purified by column chromatography on silica gel eluting with 1:3 ethyl acetate/petroleum ether to afford the title compound as an off-white solid (203 mg, 28%).

δ_{H} (d₆-DMSO, 400 MHz): 5.08 (2H, s), 5.10 (2H, s), 6.42-6.50 (3H, 6.58 (1H, d), 7.11 (1H, t), 7.20 (1H, t), 7.30-7.50 (11H, m), 7.62 (1H, s), 7.91 (1H, s), 8.38 (2H, s) and 9.53 (1H, s).

LCMS (ES⁺): 475 (MH⁺, 100%).

### Example 2

### 2,5-Bis-(3-hydroxyphenylamino)pyrimidine

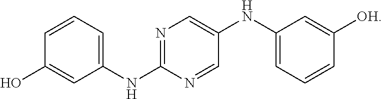 [SMILES: Oc1cccc(Nc2cnc(Nc3cccc(O)c3)nc2)c1]

1,4-Cyclohexadiene (0.20 mL, 2.11 mmol) was added to a suspension of 2,5-bis-(3-benzyloxyphenylamino)pyrimidine (48 mg, 0.10 mmol) and catalytic palladium(II) hydroxide (moist, 20% on carbon) in DMF (2 mL) and ethanol (2 mL). The suspension was heated in a sealed tube at 120° C. for 30 min under microwave irradiation at 250 W. After cooling to room temperature, the mixture was diluted with ethyl acetate and washed with water and brine. The organic phase was dried (MgSO₄) and the solvent removed under reduced pressure to afford the title compound as a brown solid (20 mg, 68%).

δ_{H} (d₆-DMSO, 400 MHz): 6.19 (1H, d), 6.32 (3H, m), 6.98 (1H, t), 7.04 (1H, t), 7.14 (1H, d), 7.35 (1H, m), 7.77 (1H, s), 8.36 (2H, s), 9.19 (2H, m), 9.37 (1H, s).

LCMS (ES⁺): 295 (MH⁺, 100%).

### Example 3

### 2-(3-Benzyloxyphenylamino)-5-bromopyrimidine

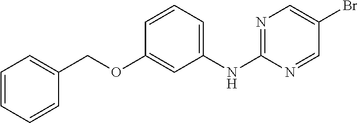 [SMILES: Brc1cnc(Nc2cccc(OCc3ccccc3)c2)nc1]

A solution of 2-chloro-5-bromopyrimidine (557 mg, 2.88 mmol) and 3-benzyloxyaniline (600 mg, 3.01 mmol) in iso-propanol (2 mL) was heated in a sealed tube at 140° C. for 1 h under microwave irradiation. After cooling to room temperature, the solvent was removed under reduced pressure and the crude product was purified by column chromatography on silica gel eluting with 1:3 ethyl acetate/petroleum ether to afford the title compound as an off-white solid (510 mg, 50%).

δ_{H} (d₆-DMSO, 400 MHz): 5.06 (2H, s), 6.65 (1H, d), 7.16 (1H, t), 7.24 (1H, d), 7.33 (1H, t), 7.40 (2H, t), 7.44 (2H, d), 7.51 (1H, s), 8.59 (2H, s) and 10.04 (1H, s).

LCMS (ES⁺): 358, 356 (MH⁺, 100%).

### Example 4

### 2-(3-Hydroxyphenylamino)-5-[3-(trifluoromethyl)phenylamino]-pyrimidine

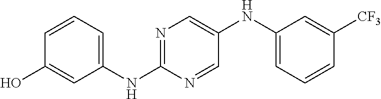 [SMILES: Oc1cccc(Nc2ncc(Nc3cccc(C(F)(F)F)c3)cn2)c1]

A suspension of 2-(3-benzyloxyphenylamino)-5-bromopyrimidine (82 mg, 0.23 mol), 3-(trifluoromethyl)aniline (148 mg, 0.92 mmol), tris(dibenzylideneacetone)-palladium(0) (10 mg, 10.9 μmol), 4,5-bis(diphenylphosphino)-9,9-dimethylxanthene (12 mg, 20.7 μmol) and cesium carbonate (90 mg, 0.28 mmol) in degassed 1,4-dioxan (2 mL) was heated at 85° C. for 3 days. After cooling to room temperature, the mixture was diluted with ethyl acetate and washed with water and brine. The organic phase was dried (MgSO₄) and the solvent removed under reduced pressure to give a brown oil. The crude product was purified by column chromatography on silica gel eluting with 1:3 ethyl acetate/petroleum ether to afford the intermediate 2-(3-benzyloxyphenylamino)-5-[3-(trifluoromethylphenyl)amino]pyrimidine.

1,4-Cyclohexadiene (50 μL, 0.52 mmol) was added to a suspension of 2-(3-benzyloxyphenylamino)-5-[3-(trifluoromethylphenyl)amino]pyrimidine (91.5 μmol) and catalytic palladium(II) hydroxide (moist, 20% on carbon) in methanol (2 mL). The suspension was heated in a sealed tube at 100° C. for 30 min under microwave irradiation at 250 W. After cooling to room temperature, the mixture was diluted with ethyl acetate and washed with water and brine. The organic phase was dried (MgSO₄) and the solvent removed under reduced pressure to give a brown oil. This crude product was purified by column chromatography on silica gel eluting with ethyl acetate/petroleum ether to give the target compound.

δ_{H} (d₆-DMSO, 400 MHz): 6.31 (1H, d), 6.97-7.03 (2H, m), 7.05 (1H, d), 7.10 (1H, d), 7.30 (1H, m), 7.35 (1H, t), 8.20 (1H, s), 8.38 (2H, s), 9.20 (1H, s), 9.44 (1H, s).

LCMS (ES⁺): 347 (MH⁺, 100%)

### Example 5

### 2-(3-Hydroxyphenylamino)-5-[3,4-dichlorophenylamino]-pyrimidine

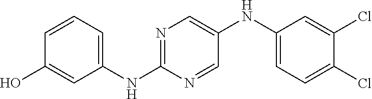 [SMILES: Oc1cccc(Nc2ncc(Nc3ccc(Cl)c(Cl)c3)cn2)c1]

A suspension of 2-(3-benzyloxyphenylamino)-5-bromopyrimidine (82 mg, 0.23 mol), 3,4-dichloroaniline (148 mg, 0.92 mmol), tris(dibenzylideneacetone)-palladium(0) (10 mg, 10.9 μmol) 4,5-bis(diphenylphosphino)-9,9-dimethylxanthene (12 mg, 20.7 μmol) and cesium carbonate (90 mg, 0.28 mmol) in degassed 1,4-dioxan (2 mL) was heated at 85° C. for 3 days. After cooling to room temperature, the mixture was diluted with ethyl acetate and washed with water and brine. The organic phase was dried (MgSO₄) and the solvent removed under reduced pressure to give a brown oil. The crude product was purified by column chromatography on silica gel eluting with 1:3 ethyl acetate/petroleum ether to afford the intermediate 2-(3-benzyloxyphenylamino)-5-(3,4-dichlorophenylamino)pyrimidine.

1,4-Cyclohexadiene (50 μL, 0.52 mmol) was added to a suspension of 2-(3-benzyloxyphenylamino)-5-(3,4-dichlorophenylamino)pyrimidine (91.5 μmol) and catalytic palladium(II) hydroxide (moist, 20% on carbon) in methanol (2 mL). The suspension was heated in a sealed tube at 100° C. for 30 min under microwave irradiation at 250 W. After cooling to room temperature, the mixture was diluted with ethyl acetate and washed with water and brine. The organic phase was dried (MgSO₄) and the solvent removed under reduced pressure to give a brown oil. This crude product was purified by column chromatography on silica gel eluting with ethyl acetate/petroleum ether to give the title compound.

δ_{H} (d₆-DMSO, 400 MHz): 6.35 (1H, d), 6.80 (1H, d), 6.97 (1H, d), 7.03 (1H, t), 7.13 (1H, d), 733 (1H, m), 7.37 (1H, d), 8.19 (1H, s), 8.38 (2H, s), 9.24 (1H, bs), 9.48 (1H, s).

LCMS (ES⁺): 347 (MH⁺, 100%).

### Example 6

### 2-(3-Trifluoromethylphenylamino)-5-bromopyrimidine

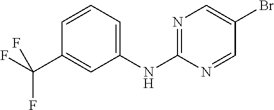 [SMILES: FC(F)(F)c1cccc(Nc2ncc(Br)cn2)c1]

A solution of 2-chloro-5-bromopyrimidine (0.50 g, 2.58 mmol) and 3-(trifluoro-methyl)aniline (0.38 mL, 3.10 mmol) in n-butanol (10 mL) was heated in a sealed tube at 120° C. for 18 h. After cooling to room temperature, scavenger resin (4-benzyloxy-benzaldehyde, polymer-bound) was added to remove excess aniline and the suspension was stirred overnight. The resin was filtered off and the solvent removed from the filtrate under reduced pressure to afford the target compound as a brown oil (0.78 g, 95%).

δ_{H} (d₆-DMSO, 400 MHz): 7.34 (1H, d), 7.56 (1H, t), 8.00 (1H, d), 8.20 (1H, s), 8.71 (2H, s) and 10.23 (1H, s).

LCMS (ES⁺): 320, 318 (MH⁺, 100%).

### Example 7

### 2-(3-Trifluoromethylphenylamino)-5-(3-benzyloxyphenylamino)-pyrimidine

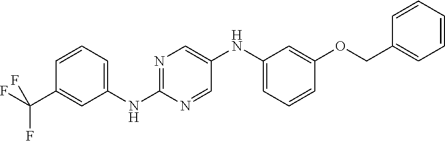 [SMILES: FC(F)(F)c1cccc(Nc2ncc(Nc3cccc(OCc4ccccc4)c3)cn2)c1]

A suspension of 2-(3-trifluoromethylphenylamino)-5-bromopyrimidine (200 mg, 0.63 mmol), 3-benzyloxyaniline (280 mg, 1.38 mmol), tris(dibenzylideneacetone) palladium(0) (23 mg, 25.0 μmol), 4,5-bis(diphenylphosphino)-9,9-dimethylxanthene (29 mg, 50.3 μmol) and cesium carbonate (530 mg, 1.50 mmol) in degassed 1,4-dioxan (5 mL) was heated at 100° C. for 24 h. After cooling to room temperature, the mixture was diluted with ethyl acetate and washed with water and brine. The organic phase was dried (MgSO₄) and the solvent removed under reduced pressure. The crude product was purified by column chromatography on silica gel eluting with 1:6 ethyl acetate/petroleum ether to afford the target compound as a yellow solid (71 mg, 25%). δ_{H} (d₆-DMSO, 400 MHz): 5.09 (2H, s), 6.47 (1H, dd), 6.53 (2H, m), 7.13 (1H, t), 7.26 (1H, d), 7.36 (1H, m), 7.40-7.47 (4H, t), 7.53 (1H, t), 8.01 (1H, s), 8.04 (1H, d), 8.24 (1H, s), 8.42 (2H,$) and 8.90 (1H, s).

LCMS (AP⁺): 437 (MH⁺, 100%).

### Example 8

### 2-(3-Trifluoromethylphenylamino)-5-(3-hydroxyphenylamino)-pyrimidine

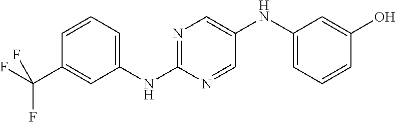 [SMILES: Oc1cccc(Nc2cnc(Nc3cccc(C(F)(F)F)c3)nc2)c1]

1,4-Cyclohexadiene (120 μL, 1.28 mmol) was added to a suspension of 2-(3-trifluoromethylphenylamino)-5-(3 benzyloxyphenylamino)-pyrimidine (58 mg, 0.128 mmol) and catalytic palladium(II) hydroxide (moist, 20% on carbon) in ethyl acetate (2 mL). The suspension was heated in a sealed tube at 110° C. for 35 min under microwave irradiation at 250 W. After cooling to room temperature, the mixture was diluted with ethyl acetate and filtered. The solvent was removed under reduced pressure to afford the target compound as a brown solid (40 mg, 90%).

δ_{H} (d₆-DMSO, 400 MHz): 6.22 (1H, d), 6.38 (2H, d), 7.01 (1H, t), 7.25 (1H, d), 7.52 (1H, t), 7.91 (1H, s), 8.03 (1H, d), 8.24 (1H, s), 8.43 (2H, s), 9.22 (1H, br s) and 9.89 (1H, s).

LCMS (ES⁺): 347 (MH⁺, 100%).

### Example 9

### 2-(3,4-Dichlorophenylamino)-5-bromopyrimidine

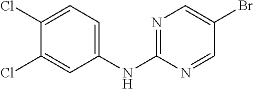 [SMILES: Clc1ccc(Nc2ncc(Br)cn2)cc1Cl]

This was prepared in an identical manner to Example 6 using 2-chloro-5-bromo-pyrimidine (0.50 g, 2.58 mmol) and 3,4-dichloroaniline (0.50 g, 3.10 mmol) to afford the target compound as a brown oil (0.57 g, 72%).

δ_{H} (d₆-DMSO, 400 MHz): 7.57 (1H, d), 7.69 (1H, dd), 8.14 (1H, d), 8.71 (2H, s) and 10.22 (1H, s).

LCMS (AP⁺): 320 (MH⁺, 100%).

### Example 10

### 2-(3,4-Dichlorophenylamino)-5-(3-methoxyphenylamino)-pyrimidine

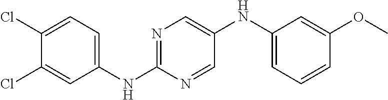 [SMILES: COc1cccc(Nc2cnc(Nc3ccc(Cl)c(Cl)c3)nc2)c1]

This was prepared in an identical manner to Example 7 using 2-(3,4-dichlorophenyl-amino)-5-bromopyrimidine (200 mg, 0.63 mmol) and 3-methoxyaniline (160 mg, 1.38 mmol) to afford the target compound as a brown solid (42 mg, 19%).

δ_{H} (d₆-DMSO, 400 MHz): 3.74 (3H, s), 6.38 (1H, d), 6.44 (1H, d), 6.52 (1H, d), 7.13 (1H, t), 7.52 (1H, d), 7.70 (1H, d), 8.03 (1H, s), 8.19 (1H, d), 8.46 (2H, s) and 9.89 (1H, s).

LCMS (ES⁺): 361 (MH⁺, 100%).

### Example 11

### 2-(3-Hydroxyphenylamino)-5-[phenyl(methyl)amino]pyrimidine

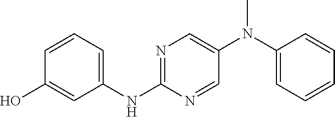 [SMILES: CN(c1ccccc1)c1cnc(Nc2cccc(O)c2)nc1]

A suspension of 2-(3-benzyloxyphenylamino)-5-bromopyrimidine (50 mg, 0.14 mmol), N-methylaniline (20 μg, 0.168 mmol), tris(dibenzylideneacetone)palladium(0) (2.6 mg, 2.8 μmol), 2-dicyclohexylphosphino-2′,4′,6′-triisopropylbiphenyl (XPhos, 5.3 mg, 11.2 μmol) and sodium tert-butoxide (20 mg, 0.19 mmol) in degassed toluene (1 mL) was heated at 100° C. for 24 h. After cooling to room temperature, the mixture was diluted with ethyl acetate and washed with water and brine. The organic phase was dried (MgSO₄) and the solvent removed under reduced pressure. The crude product was purified by column chromatography on silica gel eluting with 1:4 ethyl acetate/petroleum ether to afford the intermediate benzyl ether as a brown oil (27 mg, 50%).

1,4-Cyclohexadiene (70 μL, 0.70 mmol) was added to a suspension of the intermediate benzyl ether (27 mg, 70 μmol) and catalytic palladium(II) hydroxide (moist, 20% on carbon) in ethyl acetate (1 mL). The suspension was heated in a sealed tube at 110° C. for 20 min under microwave irradiation at 250 W. After cooling to room temperature, the mixture was diluted with ethyl acetate and filtered. The solvent was removed under reduced pressure and the crude product was purified by column chromatography on silica eluting with ethyl acetate/petroleum ether to afford the target compound as a yellow oil (10 mg, 48%).

δ_{H} (d₆-DMSO, 400 MHz): 3.27 (3H, s), 6.37 (1H, dd), 6.82 (4H, in), 7.06 (1H, t), 7.17 (1H, d), 7.24 (1H, t), 7.35 (1H, d), 8.40 (2H, s), 9.28 (1H, s) and 9.61 (1H, s).

LCMS (ES⁺): 293 (MH⁺, 100%).

### Example 12

### 2-(3-Benzyloxyphenyl-N-tert-butyloxycarbonylamino)-5-bromopyrimidine

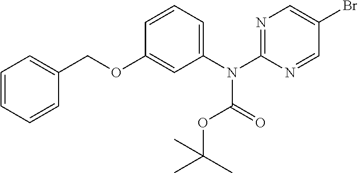 [SMILES: CC(C)(C)OC(=O)N(c1cccc(OCc2ccccc2)c1)c1ncc(Br)cn1]

Di-tert-butyldicarbonate (0.56 g, 2.59 mmol) was added to a solution of 2-(3-benzyloxyphenylamino)-5-bromopyrimidine (0.77 g, 2.16 mmol), pyridine (0.35 mL, 4.34 mmol) and DMAP (26 mg, 0.21 mmol) in THF (10 mL) and the mixture was heated at 60° C. for 18 h. After cooling to room temperature, the mixture was diluted with diethyl ether and washed with aqueous hydrochloric acid (0.5 M) and brine. The solution was dried (MgSO₄) and the solvent removed under reduced pressure. The residual solid was washed with hexane and dried to afford the target compound as a pale brown solid (0.74 g, 75%).

δ_{H} (CDCl₃, 400 MHz): 1.44 (9H, s), 5.04 (2H, s), 6.79-6.83 (2H, m), 6.93 (1H, dd), 7.28-7.42 (6H, m) and 8.64 (2H, s).

LCMS (ES⁺): 458, 456 ([M-Boc]⁺, 10%), 358, 356 (MH⁺, 100).

### Example 13

### 2-(3-Hydroxyphenylamino)-5-[3-trifluoromethylphenyl(methyl)-amino]pyrimidine

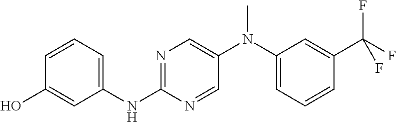 [SMILES: CN(c1cnc(Nc2cccc(O)c2)nc1)c1cccc(C(F)(F)F)c1]

The reaction between 2-(3-benzyloxyphenyl-N-tert-butyloxycarbonylamino)-5-bromopyrimidine (130 mg, 0.28 mmol) and N-methyl-3-trifluoromethylaniline (100 mg, 0.57 mmol) was carried out in an identical manner to that used for Example 7 to afford the coupled product as a colourless oil (59 mg, 44%).

Trifluoroacetic acid (2 mL) was added to a solution of the coupled product (60 mg, 0.11 mmol) in dichloromethane (2 mL). The solution was stirred at room temperature for 70 min and the solvents were removed under reduced pressure. The residual oil was redissolved in diethyl ether and the solution was washed with saturated aqueous sodium hydrogen carbonate solution, dried (MgSO₄), and the solvent removed under reduced pressure to afford the intermediate benzyl ether as a pale brown oil (52 mg).

1,4-Cyclohexadiene (110 μL, 1.16 mmol) was added to a suspension of the intermediate benzyl ether (52 mg, 0.115 mmol) and catalytic palladium(II) hydroxide (moist, 20% on carbon) in methanol (2 mL). The suspension was heated in a sealed tube at 110° C. for 50 min under microwave irradiation at 250 W. After cooling to room temperature, the mixture was diluted with ethyl acetate and filtered. The solvent was removed under reduced pressure and the crude product was purified by column chromatography on silica eluting with 1:2 ethyl acetate/petroleum ether to afford the target compound as a pale brown solid (24 mg, 58% from the coupled product).

δ_{H} (d_{d}-DMSO, 400 MHz): 3.33 (3H, s), 6.40 (1H, dd), 6.98 (1H, s), 7.01 (1H, d), 7.08 (2H, t), 7.18 (1H, dd), 7.36 (1H, d), 7.42 (1H, t), 8.47 (2H, s), 9.28 (1H, s) and 9.68 (1H, s).

LCMS (ES⁺): 361 (MH⁺, 100%).

### Example 14

### 2-(3-Benzyloxyphenyl-N-tert-butyloxycarbonylamino)-5-hydroxypyrimidine

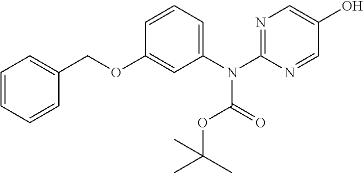 [SMILES: CC(C)(C)OC(=O)N(c1cccc(OCc2ccccc2)c1)c1ncc(O)cn1]

A mixture of 2-(3-benzyloxyphenyl-N-tert-butyloxycarbonylamino)-5-bromo-pyrimidine (2.70 g, 5.92 mmol), bis(pinacolato)diborane (2.25 g, 8.86 mmol), [1,1′-bis(diphenylphosphino)ferrocene]dichloropalladium(II) (0.24 g, 0.29 mmol) and potassium acetate (1.74 g, 17.7 mol) in degassed DMSO (75 mL) was heated at 80° C. under nitrogen for 2 h. The mixture was cooled to room temperature and diluted with ethyl acetate/diethyl ether. The suspension was washed with water (×2) and brine, dried (MgSO₄), and the solvent removed under reduced pressure. The crude product was triturated with diethyl ether and filtered to remove insoluble biaryl impurities. The solvent was removed from the filtrate and the residue triturated with diisopropyl ether to afford the intermediate boronic ester as a pale yellow solid (1.41 g, 47%).

The intermediate boronic ester (0.80 g, 1.59 mmol) was suspended in methanol (12 mL) and aqueous hydrogen peroxide (27%, 0.65 mL, 5.13 mmol) was added. The mixture was stirred at room temperature for 1 h. The resulting solution was concentrated under reduced pressure and the residual oil was redissolved in diethyl ether. The solution was washed with water (×2) and brine, dried (MgSO₄), and the solvent removed under reduced pressure to afford the target compound as a pale brown oil (0.58 g, 92%).

δ_{H} (CDCl₃, 400 MHz): 1.44 (9H, s), 5.01 (2H, s), 6.83-6.87 (3H, m), 7.23 (1H, t), 7.32-7.40 (5H, m), 7.56 (1H, br s) and 8.16 (2H, s).

LCMS (ES⁺): 394 ([M-Boc]⁺, 100%).

### Example 15

### 2-(3-Benzyloxyphenylamino)-5-(4-fluorophenoxy)pyrimidine

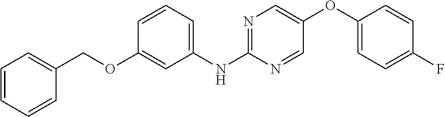 [SMILES: Fc1ccc(Oc2cnc(Nc3cccc(OCc4ccccc4)c3)nc2)cc1]

A mixture of 2-(3-benzyloxyphenyl-N-tert-butyloxycarbonylamino)-5-hydroxypyrimidine (110 mg, 0.28 mmol), 4-fluorophenylboronic acid (58 mg, 0.41 mmol), copper (II) acetate (51 mg, 0.28 mmol), triethylamine (195 μL, 1.40 mmol) and powdered 4 Å molecular sieves in dichloromethane (3 mL) was stirred under air for 3 days. The suspension was diluted with ethyl acetate, filtered, and washed with water and brine. The solution was dried (MgSO₄) and the solvent removed under reduced pressure. The crude product was purified by column chromatography on silica eluting with 1:5 ethyl acetate/petroleum ether to afford the intermediate Boc-protected amine as a yellow oil (30 mg, 22%).

The intermediate (30 mg) was dissolved in dichloromethane (5 mL) and trifluoroacetic acid (2 mL) was added. The solution was stirred at room temperature for 1 h, then diluted with dichloromethane and washed with saturated aqueous sodium hydrogen carbonate solution. The organic phase was dried (MgSO₄) and the solvent removed under reduced pressure. The crude product was purified by column chromatography on silica eluting with 1:5 ethyl acetate/petroleum ether to afford the target compound as a yellow oil (15 mg, 63%).

δ_{H} (CDCl₃, 400 MHz): 5.10 (2H, s), 6.62 (1H, dd), 7.10-7.14 (2H, m), 7.17-7.26 (3H, m), 7.30 (1H, dd), 7.37 (1H, d), 7.42 (2H, t), 7.48 (2H, m), 7.61 (1H, t), 8.44 (2H, s) and 9.74 (1H, s).

LCMS (AP⁺): 388 (MH⁺, 100%).

### Example 16

### 2-(3-Hydroxyphenylamino)-5-(4-fluorophenoxy)pyrimidine

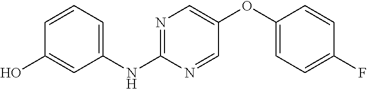 [SMILES: Oc1cccc(Nc2ncc(Oc3ccc(F)cc3)cn2)c1]

1,4-Cyclohexadiene (150 μL, 1.58 mmol) was added to a suspension of 2-(3, benzyloxyphenylamino)-5-(4-fluorophenoxy)pyrimidine (41 mg, 0.106 mmol) and catalytic palladium(II) hydroxide (moist, 20% on carbon, 10 mg) in ethyl acetate (2 mL). The suspension was heated in a sealed tube at 110° C. for 1 h under microwave irradiation at 250 W. After cooling to room temperature, the mixture was diluted with ethyl acetate and filtered. The solvent was removed under reduced pressure to afford the title compound as a brown foam (30 mg, 94%).

δ_{H} (d₆-DMSO, 400 MHz): 6.38 (1H, dd), 7.06 (1H, t), 7.10-7.17 (3H, m), 7.21-7.26 (2H, m), 7.33 (1H, d), 8.42 (2H, s), 9.26 (1H, s) and 9.62 (1H, s).

LCMS (ES⁺): 298 (MH⁺, 100%).

### Example 17

### 2-(3-Hydroxyphenylamino)-5-[4-fluorophenyl(methyl)amino]pyrimidine

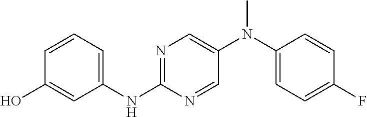 [SMILES: CN(c1ccc(F)cc1)c1cnc(Nc2cccc(O)c2)nc1]

This was prepared in an identical manner to Example 14, using 4-fluoro-N-methylaniline in the palladium-catalysed reaction to afford the target compound as a yellow solid.

δ_{H} (d₆-DMSO, 400 MHz) 3.26 (3H, s), 6.38 (1H, m), 6.87 (2H, m), 7.01-7.18 (3H, m), 7.32 (1H, s), 8.43 (2H, s), 9.22 (1H, br. s) and 9.52 (1H, s).

LCMS (ES⁺): 311 (MH⁺, 100%).

### Example 18

### 5-Bromo-2-(N-tert-butyloxycarbonylphenylamino)pyrimidine

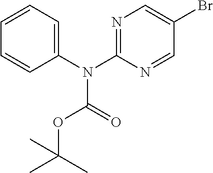 [SMILES: CC(C)(C)OC(=O)N(c1ccccc1)c1ncc(Br)cn1]

The chloride displacement reaction was performed as for Example 6 starting from 2-chloro-5-bromopyrimidine (2.00 g, 10.3 mmol) and aniline (0.95 mL, 10.4 mmol). Boc-protection was performed as for Example 13 to afford the target compound as a yellow oil (120 mg, 36%).

### Example 19

### 5-[4-Fluorophenyl(methyl)amino]2-(phenylamino)-pyrimidine

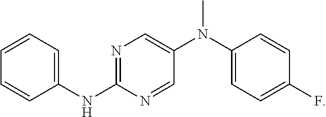 [SMILES: CN(c1ccc(F)cc1)c1cnc(Nc2ccccc2)nc1]

The palladium-catalysed reaction between 2-(N-tert-butyloxycarbonylphenylamino)-5-bromopyrimidine (120 mg, 0.34 mmol) and N-methyl-3-trifluoromethylaniline (72 μL, 0.60 mmol) was carried out as for Example 7 to afford the coupled material which was redissolved in dichloromethane (1 mL) and trifluoroacetic acid (0.5 mL). The solution was stirred at room temperature overnight and the solvents were removed under reduced pressure. The residual oil was redissolved in diethyl ether and the solution was washed with saturated aqueous sodium hydrogen carbonate solution, dried (MgSO₄), and the solvent removed under reduced pressure to afford the target compound as an off-white solid (25 mg, 25%).

δ_{H} (d₆-DMSO, 400 MHz) 3.26 (3H, s), 6.38 (1H, m), 6.85 (2H, m), 6.95 (1H, t), 7.07 (2H, t), 7.28 (2H, t), 7.78 (2H, d), 8.45 (2H, s) and 9.64 (1H, s).

LCMS (ES⁺): 295 (MH⁺, 100%).

### Example 20

### 5-[4-Fluorophenyl(methyl)amino]-2-(3-methoxyphenylamino)pyrimidine

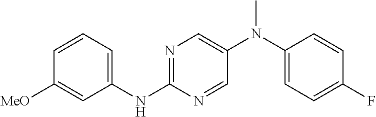 [SMILES: COc1cccc(Nc2ncc(N(C)c3ccc(F)cc3)cn2)c1]

This was prepared in an identical manner to that used for 5-[4-fluorophenyl(methyl)amino]2-(phenylamino)-pyrimidine starting from 2-chloro-5-bromopyrimidine and m-anisidine to afford the target compound as a brown gum (17 mg, 25% overall yield).

δ_{H} (d₆-DMSO, 400 MHz) 3.26 (3H, s), 3.76 (3H, s), 6.52 (1H, m), 6.89 (2H, m), 7.09 (2 H t), 7.14 (1H, t), 7.32 (1H, d), 7.50 (1H, d), 8.47 (2H, s), and 9.62 (1H, s).

LCMS (AP⁺): 325 (MH⁺, 100%).

### Example 21

### 2-(3-Fluorophenylamino)5-[4-fluorophenyl(methyl)amino]pyrimidine

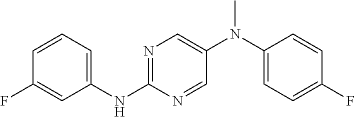 [SMILES: CN(c1ccc(F)cc1)c1cnc(Nc2cccc(F)c2)nc1]

This was prepared in an identical manner to that used for 5-[4-fluorophenyl(methyl)amino]2-(phenylamino)-pyrimidine starting from 2-chloro-5-bromopyrimidine and 3-fluoroaniline to afford the target compound as a brown gum (78 mg, 69% overall yield).

δ_{H} (d₆-DMSO, 400 MHz) 3.27 (3H, s), 6.72 (1H, m), 6.91 (2H, m), 7.09 (2H, t), 7.38 (1H, q), 7.48 (1H, d), 7.81 (1H, d), 8.39 (2H, s), and 9.89 (1H, s).

LCMS (AP⁺): 313 (MH⁺, 100%).

The following compounds were prepared in an similar manner to that described for 2-(3-hydroxyphenylamino)-5-(4-fluorophenoxy)pyrimidine starting from 2-(3-benzyloxy-phenyl-N-tert-butyloxycarbonylamino)-5-hydroxypyrimidine and the appropriate arylboronic acid:

### Example 22

### 2-(3-Hydroxyphenylamino)-5-(3-methoxyphenoxy)pyrimidine

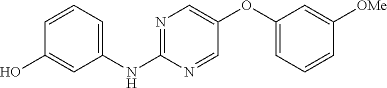 [SMILES: COc1cccc(Oc2cnc(Nc3cccc(O)c3)nc2)c1]

δ_{H} (d₆-DMSO, 400 MHz) 3.78 (3H, s), 6.38 (1H, m), 6.56 (1H, m), 6.62 (1H, m), 6.72 (1H, m), 7.03 (1H, t), 7.13 (1H, m), 7.26 (1H, t), 7.33 (1H, m), 8.41 (2H, s), 9.26 (1H, br. s) and 9.63 (1H, s).

LCMS (AP⁺): 310 (MH⁺, 100%).

### Example 23

### 2-(3-Hydroxyphenylamine)-5-(4-methylsulfonylphenoxy)pyrimidine

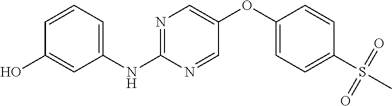 [SMILES: CS(=O)(=O)c1ccc(Oc2cnc(Nc3cccc(O)c3)nc2)cc1]

δ_{H} (d₆-DMSO, 400 MHz) 3.21 (3H, s), 6.38 (1H, m), 7.03 (1H, t), 7.13 (1H, m), 7.25 (2H, d), 7.33 (1H, m), 7.99 (2H, d), 8.51 (2H, s), 9.29 (1H, br. s) and 9.73 (1H, s).

LCMS (AP⁺): 358 (MH⁺, 100%).

### Example 24

### 2-(3-Hydroxyphenylamino)-5-(4-methylaminosulfonylphenoxy)pyrimidine

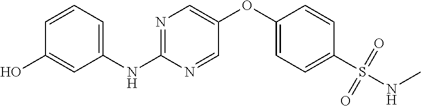 [SMILES: CNS(=O)(=O)c1ccc(Oc2cnc(Nc3cccc(O)c3)nc2)cc1]

δ_{H} (d₆-DMSO, 400 MHz) 2.44 (3H, d), 6.38 (1H, m), 7.03 (1H, t), 7.13 (1H, m), 7.21 (2H, d), 7.31 (1H, m), 7.40 (1H, q), 7.78 (2H, d), 8.52 (2H, s), 9.28 (1H, br. s) and 9.72 (1H, s).

LCMS (ES⁺): 373 (MH⁺, 100%).

### Example 25

### 2-(3-Hydroxyphenylamino)-5-(3-dimethylaminophenoxy)pyrimidine

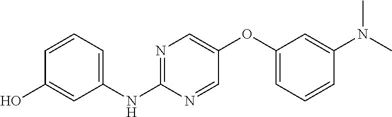 [SMILES: CN(C)c1cccc(Oc2cnc(Nc3cccc(O)c3)nc2)c1]

δ_{H} (d₆-DMSO, 400 MHz) 2.92 (6H, s), 6.22 (1H, m), 6.38 (2H, m), 6.50 (1H, m), 7.03 (1H, t), 7.13 (2H, m), 7.34 (1H, m), 8.38 (2H, s), 9.25 (1H, br. s) and 9.58 (1H, s).

LCMS (AP⁺): 323 (MH⁺, 100%).

### Example 26

### 2-(3-Hydroxyphenylamino)-5-(2,3-dihydrobenzo[1,4]dioxin-6-yl)oxypyrimidine

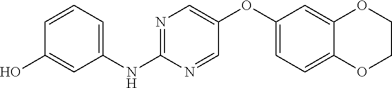 [SMILES: Oc1cccc(Nc2ncc(Oc3ccc4c(c3)OCCO4)cn2)c1]

δ_{H} (d₆-DMSO, 400 MHz) 4.26 (4H, m), 6.38 (1H, m), 6.54 (1H, m), 6.61 (1H, m), 6.87 (1H, d), 7.05 (1H, t), 7.13 (1H, m), 7.32 (1H, m), 8.36 (2H, s), 9.25 (1H, br. s) and 9.57 (1H, s).

LCMS (AP⁺): 338 (MH⁺, 100%).

### Example 27

### 2-(3-Hydroxyphenylamino)-5-[3-(pyrrolidin-1-yl)phenoxy]pyrimidine

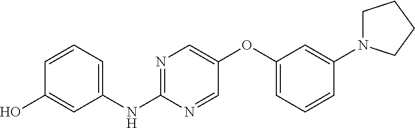 [SMILES: Oc1cccc(Nc2ncc(Oc3cccc(N4CCCC4)c3)cn2)c1]

δ_{H} (d₆-DMSO, 400 MHz) 1.97 (4H, m), 3.22 (4H, m), 6.17 (2H, m), 6.32 (1H, m), 6.37 (1H, m), 7.03 (1H, t), 7.13 (2H, m), 7.34 (1H, m), 8.38 (2H, s), 9.25 (1H, br. s) and 9.58 (1H, s).

LCMS (AP⁺): 349 (MH⁺, 100%).

### Example 28

### 5-(3-Benzyloxyphenoxy)-2-(3-hydroxyphenylamino)pyrimidine

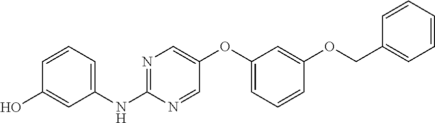 [SMILES: Oc1cccc(Nc2ncc(Oc3cccc(OCc4ccccc4)c3)cn2)c1]

δ_{H} (d₆-DMSO, 400 MHz) 5.13 (2H, s), 6.38 (1H, m), 6.58 (1H, m), 6.70 (1H, m), 6.78 (1H, m), 7.03 (1H, t), 7.13 (1H, m), 7.27-7.47 (7H, m), 8.42 (2H, s), 9.29 (1H, br. s) and 9.66 (1H, s).

LCMS (ES⁺): 386 (MH⁺, 100%).

### Example 29

### 5-Bromo-2-(phenylamino)pyrimidine

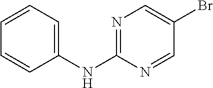 [SMILES: Brc1cnc(Nc2ccccc2)nc1]

A solution of 2-chloro-5-bromopyrimidine (2.00 g, 10.3 mmol) and aniline (0.95 mL, 10.4 mmol) in n-butanol (10 mL) was heated in a sealed tube at 110° C. for 18 h. The solvent was removed under reduced pressure to afford 5-Bromo-2-(phenylamino)pyrimidine as a brown solid (2.95 g).

### Example 30

### 2-(Phenylamino)-5-(4,4,5,5-tetramethyl-[1,3,2]dioxaborolan-2-yl)pyrimidine

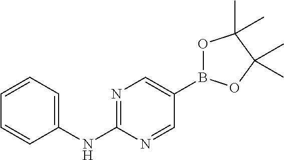 [SMILES: CC1(C)OB(c2cnc(Nc3ccccc3)nc2)OC1(C)C]

A mixture of 5-bromo-2-(phenylamino)pyrimidine (2.00 g, ˜8.0 mmol), bis(pinacolato)diborane (2.40 g, 9.44 mmol), [1,1′-bis(diphenylphosphino)ferrocene]dichloropalladium(II) (0.32 g, 0.39 mmol) and potassium acetate (2.35 g, 23.9 mmol) in degassed DMSO (20 mL) was heated at 80° C. under nitrogen for 2 h. The mixture was cooled to room temperature and diluted with ethyl acetate/diethyl ether. The suspension was washed with water (×2) and brine, dried (MgSO₄), and the solvent removed under reduced pressure to afford the crude boronic ester as a brown solid (2.84 g).

### Example 31

### 5-Hydroxy-2-(phenylamino)pyrimidine

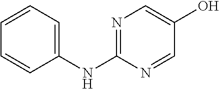 [SMILES: Oc1cnc(Nc2ccccc2)nc1]

2-(Phenylamino)-5-(4,4,5,5-tetramethyl-[1,3,2]dioxaborolan-2-yl)pyrimidine (2.84 g) was suspended in methanol (50 mL) and aqueous hydrogen peroxide (27%, 3 mL, 24 mmol) was added. The mixture was stirred at room temperature for 1 h. The resulting solution was concentrated under reduced pressure and partitioned between ethyl acetate and aqueous sodium hydroxide solution (0.7 M). The basic aqueous extract was acidified to pH 5 (0.5 M HCl) and re-extracted with ethyl acetate. The extract was washed with brine, dried (MgSO₄), and the solvent removed under reduced pressure to afford 5-hydroxy-2-(phenylamino)pyrimidine as a brown oil (0.30 g) which was used directly in the next step.

### Example 32

### 5-(4-Fluorophenoxy)-2-(phenylamino)pyrimidine

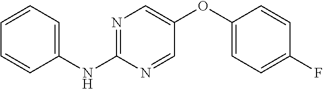 [SMILES: Fc1ccc(Oc2cnc(Nc3ccccc3)nc2)cc1]

A mixture of the 5-hydroxy-2-(phenylamino)pyrimidine (100 mg, ˜0.53 mmol), 4-fluorophenylboronic acid (150 mg, 1.07 mmol), copper (II) acetate (115 mg, 0.63 mmol), triethylamine (370 μL, 2.66 mmol) and powdered 4 Å molecular sieves in dichloromethane (5 mL) was stirred under air for 3 days. The suspension was diluted with ethyl acetate, filtered, and washed with water and brine. The solution was dried (MgSO₄) and the solvent removed under reduced pressure. The crude product was purified by column chromatography on silica (1:6 ethyl acetate/hexane) followed by removal of non-basic impurities using an MP-TsOH cartridge to afford the target compound as a brown solid (9 mg).

δ_{H} (d₆-DMSO, 400 MHz) 6.96 (1H, t), 7.10 (2H, m), 7.24 (2H, t), 7.28 (2H, t), 7.74 (2H, d), 8.41 (2H, s) and 9.71 (1H, s).

LCMS (ES⁺): 282 (MH⁺, 100%).

The following compounds were prepared in an identical manner to 5-(4-fluorophenoxy)-2-(phenylamino)pyrimidine starting from 2-chloro-5-bromopyrimidine and the appropriate anilines and boronic acids:

### Example 33

### 5-(3-Dimethylaminophenoxy)-2-(phenylamino)pyrimidine

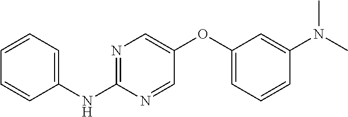 [SMILES: CN(C)c1cccc(Oc2cnc(Nc3ccccc3)nc2)c1]

δ_{H} (d₆-DMSO, 400 MHz) 2.92 (6H, s), 6.21 (1H, m), 6.38 (1H, m), 6.50 (1H, m), 6.96 (1H, t), 7.16 (1H, t), 7.29 (2H, t), 7.77 (2H, d), 8.41 (2H, s) and 9.71 (1H, s).

LCMS (AP⁺): 307 (MH⁺, 100%).

### Example 34

### 5-(2,3-Dihydrobenzo[1,4]dioxin-6-yl)oxy-2-(phenylamino)pyrimidine

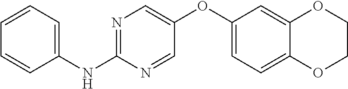 [SMILES: c1ccc(Nc2ncc(Oc3ccc4c(c3)OCCO4)cn2)cc1]

δ_{H} (d₆-DMSO, 400 MHz) 4.24 (4H, m), 6.56 (1H, m), 6.62 (1H, m), 6.87 (1H, d), 6.96 (1H, t), 7.30 (2H, t), 7.76 (2H, d), 8.38 (2H, s) and 9.68 (1H, s).

LCMS (ES⁺): 322 (MH⁺, 100%).

### Example 35

### 5-(4-Fluorophenoxy)-2-(3-methoxyphenylamino)pyrimidine

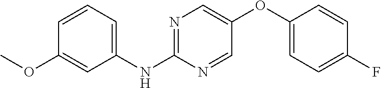 [SMILES: COc1cccc(Nc2ncc(Oc3ccc(F)cc3)cn2)c1]

δ_{H} (d₆-DMSO, 400 MHz) 3.77 (3H, s), 6.56 (1H, m), 7.12 (2H, m), 7.22 (3H, m), 7.33 (1H, m), 7.49 (1H, m), 8.44 (2H, s) and 9.72 (1H, s).

LCMS (ES⁺): 312 (MH⁺, 100%).

### Example 36

### 5-(3-Dimethylaminophenoxy)-2-(3-methoxyphenylamino)pyrimidine

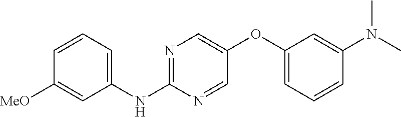 [SMILES: COc1cccc(Nc2ncc(Oc3cccc(N(C)C)c3)cn2)c1]

δ_{H} (d₆-DMSO, 400 MHz) 2.92 (6H, s), 3.77 (3H, s), 6.23 (1H, m), 6.40 (1H, m), 6.53 (2H, m), 7.18 (2H, m), 7.34 (1H, m), 7.48 (1H, m), 8.40 (2H, s) and 9.68 (1H, s).

LCMS (ES⁺): 337 (MH⁺, 100%).

### Example 37

### 5-(2,3-Dihydrobenzo[1,4]dioxin-6-yl)oxy-2-(3-methoxyphenylamino)pyrimidine

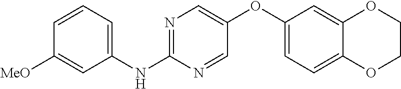 [SMILES: COc1cccc(Nc2ncc(Oc3ccc4c(c3)OCCO4)cn2)c1]

δ_{H} (d₆-DMSO, 400 MHz) 3.76 (3H, s), 4.24 (4H, m), 6.55 (2H, m), 6.63 (1H, m), 6.87 (1H, d), 7.19 (1H, t), 7.32 (1H, m), 7.48 (1H, s), 8.38 (2H, s) and 9.68 (1H, s).

LCMS (ES⁺): 352 (MH⁺, 100%).

### Example 38

### 5-(4-Fluorophenoxy)-2-(3-fluorophenylamino)pyrimidine

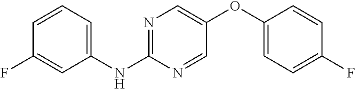 [SMILES: Fc1ccc(Oc2cnc(Nc3cccc(F)c3)nc2)cc1]

δ_{H} (d₆-DMSO, 400 MHz) 6.76 (1H, m), 7.13 (2H, m), 7.22 (2H, m), 7.32 (1H, q), 7.50 (1H, m), 7.83 (1H, m), 8.49 (2H, s) and 10.02 (1H, s).

LCMS (AP⁺): 300 (MH⁺, 100%).

### Example 39

### 5-(3-Dimethylaminophenoxy)-2-(3-fluorophenylamino)pyrimidine

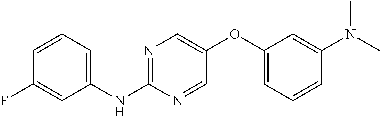 [SMILES: CN(C)c1cccc(Oc2cnc(Nc3cccc(F)c3)nc2)c1]

δ_{H} (d₆-DMSO, 400 MHz) 2.92 (6H, s), 6.23 (1H, m), 6.40 (1H, m), 6.51 (1H, m), 6.75 (1H, m), 7.17 (1H, t), 7.31 (1H, q), 7.48 (1H, m), 7.82 (1H, m), 8.45 (2H, s) and 9.98 (1H, s).

LCMS (AP⁺): 325 (MH⁺, 100%).

### Example 40

### 5-(2,3-Dihydrobenzo[1,4]dioxin-6-yl)oxy-2-(3-fluorophenylamino)pyrimidine

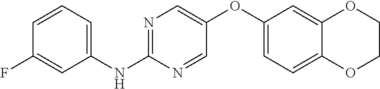 [SMILES: Fc1cccc(Nc2ncc(Oc3ccc4c(c3)OCCO4)cn2)c1]

δ_{H} (d₆-DMSO, 400 MHz) 4.26 (4H, m), 6.56 (1H, m), 6.63 (1H, m), 6.75 (1H, m), 6.88 (1H, d), 7.31 (1H, q), 7.49 (1H, s), 7.82 (1H, m), 8.43 (2H, s) and 9.97 (1H, s).

LCMS (AP⁺): 340 (MH⁺, 100%).

### Example 41

### 1-[4-(5-Bromopyrimidin-2-ylamino)phenyl]-2,2,2-trifluoroethanone

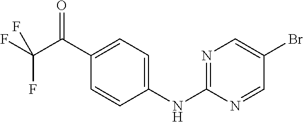 [SMILES: O=C(c1ccc(Nc2ncc(Br)cn2)cc1)C(F)(F)F]

A solution of 2-chloro-5-bromopyrimidine (300 mg, 1.58 mmol), (4-aminophenyl)-2,2,2-trifluoroethanone (300 mg, 1.58 mmol) and ammonium chloride (85 mg) in n-butanol (4 mL) was heated in a sealed tube at 110° C. for 18 h. The solvent was removed under reduced pressure and the residue washed with methanol. The methanol washings were concentrated and the resulting crude product purified by column chromatography on silica (1:4 ethyl acetate/petrol) to afford the target compound as a pale yellow solid (245 mg, 45%).

### Example 42

### 1-[4-(5-Bromopyrimidin-2-ylamino)phenyl]-2,2,2-trifluoroethanol

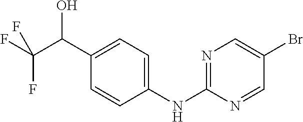 [SMILES: OC(c1ccc(Nc2ncc(Br)cn2)cc1)C(F)(F)F]

Sodium borohydride (52 mg, 1.37 mmol) was added to a solution of 1-[4-(5-bromopyrimidin-2-ylamino)phenyl]-2,2,2-trifluoroethanone (95 mg, 0.27 mmol) in methanol (5 mL). The solution was stirred at room temperature for 3 d, then diluted with ethyl acetate and washed with water and brine. The organic phase was dried (MgSO₄) and the solvent removed under reduced pressure. The crude product was purified by column chromatography an silica gel (1:4 ethyl acetate/hexane) to afford the target compound as a white solid (46 mg, 48%).

### Example 43

### 2-[4-(1-Hydroxy-2,2,2-trifluoroethyl)phenyl]amino-5-[4-fluorophenyl(methyl)amino]pyrimidine

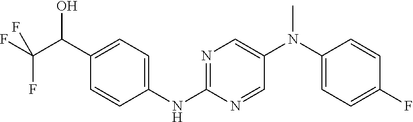 [SMILES: CN(c1ccc(F)cc1)c1cnc(Nc2ccc(C(O)C(F)(F)F)cc2)nc1]

A solution of 1-[4-(5-bromopyrimidin-2-ylamino)phenyl]-2,2,2-trifluoroethanol (46 mg, 0.13 mmol), 4-fluoro-N-methylaniline (24 μL, 0.20 mmol), 2-dicyclohexylphosphino-biphenyl (3 mg, 85 μmol), tris(dibenzylideneacetone)palladium(0) (6 mg, 6.5 μmol) and LiHMDS (1M solution in THF, 0.40 mL, 0.4 mmol) in dry THF (1.5 mL) was heated at 65° C. for 2 d. After cooling to room temperature, the mixture was diluted with ethyl acetate and washed with water and brine. The organic phase was dried (MgSO₄) and the solvent removed under reduced pressure. The crude product was purified by column chromatography on silica gel (1:4 ethyl acetate/hexane) to afford the target compound as a brown solid (9 mg, 18%).

δ_{H} (d₆-DMSO, 400 MHz) 3.26 (3H, s), 5.06 (1H, m), 6.70 (1H, d), 6.90 (2H, d), 7.09 (2H, t), 7.40 (2H, d), 7.79 (2H, d), 8.39 (2H, s) and 9.74 (1H, s).

LCMS (ES⁺): 393 (MH⁺, 100%).

### Example 44

### Preparation of Stock Solutions for Biological Assays

Ab(1-42) preparation

Aβ(1-42) was prepared for amyloid aggregation and toxicity assays by dissolving Aβ(1-42) HCl salt in hexafluoroisopropanol (HFIP), with brief sonication and vortexing. This solution of the Aβ(1-42) peptide in IMP was stored at 4° C.@2 mM. When required, an aliquot of this stock solution was freeze-dried and dissolved in DMSO to 200 times the required final assay concentration (e.g. 2 mM for a final assay concentration of 10 μM).

### Compound Preparation

A 20 mM stock solution of each test compound was prepared in DMSO, and aliquots of these solutions were used to prepare further stock solutions of each test compound in DMSO, ranging in concentration from 3 μM up to 10 mM. These stock solutions were prepared for use as and when required and stored at −20° C. (maximum of 3 freeze-thaw cycles). The 20 mM parent stock solutions were stored frozen at −20° C.

### Example 45

### Cell Viability Assay for Amyloid Toxicity Using MTT Reduction

The activity of compounds in protecting SH SY5Y cells from a toxic insult of 10 μM Aβ(1-42) was assessed by using inhibition of MTT reduction as a measure of cell viability. An aliquot (3 μl) of test compound [various concentrations] in DMSO is added to 294 μl of Opti-Mem (containing 2% FBS, 1% Pen/Strep, 1% L-Gln) {daughter plate}. The well is mixed thoroughly. Then an aliquot (3 μl) of Aβ(1-42) [2 mM] is added to the daughter plate wells and again mixed thoroughly. 50 μl is then aspirated and dispensed into wells containing 50 μl media+SH SY5Ycells (cells are also plated in Opti-Mem, at ˜30,000 cells/well/50 μl). Final concentrations of compound on cells range from [50 μM] to [˜15 nM] with a final concentration of Aβ(1-42) of [10 μM].

Cell plates are incubated for 24 h and then the MTT assay (Shearman, 1999). is performed. Briefly, 15 μl of MTT (3-(4,5-dimethylthiazol-2-yl)-2,5-diphenyl-tetrazolium bromide) dye (from Promega) added to each well and the plates incubated in 5% CO₂ at 37° C. for 4 hours. 100 μl Stop/solubilisation solution (from Promega) was added to each well and the plates were left overnight in humidified box at room temperature. The plate was shaken and the absorbance was recorded at both 570 nm and 650 nm. ΔA values were calculated by subtracting absorbance at 650 nm from absorbance at 570 nm, to reduce non-specific background absorbance. ΔA values from equivalent experiments were averaged and % cell viability was determined as follows:

$$\% cell viability = \frac{[ Δ A ( sample ) - Δ A ( dead cell control ) ] \times 100 \%}{[ Δ A ( live cell control ) - Δ A ( dead cell control ) ]}$$

Live cell controls: 1% DMSO in Opti-Mem
Dead cell controls: 0.1% Triton X-100 added to cells

The daughter plate is sealed with silver seal and incubated at 37° C. for 24 and 48 hours for the Thioflavin T assay (LeVine and Scholten 1999).

### Example 46

### Thioflavin T assay

The activity of compounds in inhibiting 10 μM Aβ(1-42) aggregation was assessed by using a thioflavin-T fluorimetric assay. At each timepoint, a 50 or 100 μl aliquot is taken from each well of the daughter plate and dispensed into a black 96 well plate. Equal volume (50 or 100 μl) Thioflavin T [40 μM] (in Glycine buffer [50 mM] —NaOH pH 8.5) is added to each well. The plate was shaken and fluorescence was recorded using the top reader setting (10×1 msec), using excitation and emission filters of 440 (±15) and 485 (±10) nm, respectively. Fluorescence readings from equivalent experiments were averaged and % amyloid formation was determined as follows:

$$\% amyloid formed = \frac{[ F ( sample ) - F ( blank ) ] \times 100 \%}{[ F ( amyloid alone ) - F ( blank ) ]} .$$

### Example 47

### Activity of Compounds in Inhibiting 10 μM Aβ(1-42) Aggregation Using Thioflavin-T Fluorimetric Assay

| | IC₅₀ (μM) |
|---|---|
| Example 2 | 32 |
| Example 4 | 13 |
| Example 8 | 5.0 |
| Example 10 | 8.5 |
| Example 11 | 22 |
| RS-0406 | 50 |

### Example 48

### Activity of Compounds in Protecting SH SY5Y Cells from a Toxic Insult of 10 μM Aβ(1-42) Using Inhibition of MTT Reduction as a Measure of Cell Viability

| | IC₅₀ (μM) |
|---|---|
| Example 2 | 14 |
| Example 11 | 30 |
| RS-0406 | 40 |

### REFERENCES

Buxbaum, J. N. (2004). “The systemic amyloidoses.” Curr Opin Rheumatol 16(1): 67-75.
Caughey, B. and P. T. Lansbury (2003). “Protofibrils, pores, fibrils, and neuro-degeneration: separating the responsible protein aggregates from the innocent bystanders.” Annu Rev Neurosci 26: 267-98.
Dev, K. K., K. Hofele, S. Barbieri, V. L. Buchman and H. van der Putten (2003). “Part II: alpha-synuclein and its molecular pathophysiological role in neurodegenerative disease.” Neuropharmacology 45(1): 14-44.
Forman, M. S., J. Q. Trojanowski and V. M. Lee (2004). “Neurodegenerative diseases: a decade of discoveries paves the way for therapeutic breakthroughs.” Nat Med 10(10): 1055-63.
Gejyo, F., T. Yamada, S. Odani, Y. Nakagawa, M. Arakawa, T. Kunitomo, H. Kataoka, M. Suzuki, Y. Hirasawa, T. Shirahama and et al. (1985). “A new form of amyloid protein associated with chronic hemodialysis was identified as beta 2-microglobulin.” Biochem Biophys Res Commun 129(3): 701-6.
Glabe, C. G. (2004). “Conformation-dependent antibodies target diseases of protein misfolding.” Trends Biochem Sci 29(10): 542-7.
Glenner, G. G. (1980a). “Amyloid deposits and amyloidosis: the beta-fibrilloses.” N Engl J Med 302(23):1283-92.
Glenner, G. G. (1980b). “Amyloid deposits and amyloidosis: the beta-fibrilloses.” N Engl J Med 302(24):1333-43.
Jaikaran, E. T. and A. Clark (2001). “Islet amyloid and type 2 diabetes: from molecular misfolding to islet pathophysiology.” Biochim Biophys Acta 1537(3): 179-203:
Kagan, B. L., Y. Hirakura, R. Azimov, R. Azimova and M. C. Lin (2002). “The channel hypothesis of Alzheimer's disease: current status.” Peptides 23(7): 1311-5.
Kayed, R., E. Head, J. L. Thompson, T. M. McIntire, S. C. Milton, C. W. Cotman and C. G. Glabe (2003). “Common structure of soluble amyloid oligomers implies common mechanism of pathogenesis.” Science 300(5618): 486-9.
LeVine, H., 3rd (2004). “The Amyloid Hypothesis and the clearance and degradation of Alzheimer's beta-peptide.” J Alzheimers Dis 6(3): 303-14.
LeVine, H., 3rd and J. D. Scholten (1999). “Screening for pharmacologic inhibitors of amyloid fibril formation.” Methods Enzymol 309: 467-76.
Masino, L (2004). “Polyglutamine and neurodegeneration: structural aspects.” Protein Pept Lett 11(3):239-48.
Ross, C. A. and M. A. Poirier (2004). “Protein aggregation and neurodegenerative disease.” Nat Med 10 Suppl: S10-7.
Selkoe, D. J. (2003). “Folding proteins in fatal ways.” Nature 426(6968): 900-4.
Shearman, M. S. (1999). “Toxicity of protein aggregates in PC12 cells: 3-(4,5-dimethylthiazol-2-yl)-2,5-diphenyltetrazolium bromide assay.” Methods Enzymol 309: 716-23.
Soto, C. and J. Castilla (2004). “The controversial protein-only hypothesis of prion propagation.” Nat Med 10 Suppl: S63-7.
Stefani, M. (2004). “Protein misfolding and aggregation: new examples in medicine and biology of the dark side of the protein world.” Biochim Biophys Acta 1739(1): 5-25.
Taylor, J. P., J. Hardy and K. H. Fischbeck (2002). “Toxic proteins in neuro-degenerative disease.” Science 296(5575): 1991-5.
Walsh, D. M., I. Klyubin, J. V. Fadeeva, W. K. Cullen, R. Anwyl, M. S. Wolfe, M. J. Rowan and D. J. Selkoe (2002). “Naturally secreted oligomers of amyloid beta protein potently inhibit hippocampal long-term potentiation in vivo.” Nature 416(6880): 535-9.
Walsh, D. M. and D. J. Selkoe (2004). “Oligomers on the brain: the emerging role of soluble protein aggregates in neurodegeneration.” Protein Pept Lett 11(3): 213-28.
Wood, J. D., T. P. Beaujeux and P. J. Shaw (2003). “Protein aggregation in motor neurone disorders.” Neuropathol Appl Neurobiol 29(6): 529-45.

1. A compound of formula (I) or a pharmaceutically acceptable salt or prodrug thereof: 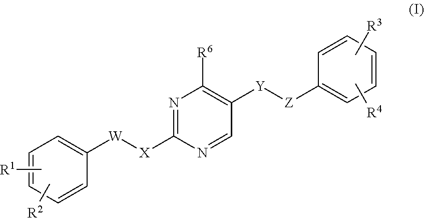
wherein
X and Y are independently NR⁵ or O;
W and Z are independently a bond or (CH₂)ₘCH(R⁷)(CH₂)ₙ;
m=0-1 and n=0-2;
R¹ and R² are independently hydrogen, halogen, CF₃, OR⁸, OR⁹, NR⁹R¹⁰, NR⁹COR¹¹, NR⁹SO₂R¹¹, SO₂NR⁹R¹⁰, SO₂R¹¹ or C₁₋₆ alkyl optionally and independently substituted by one or more of hydroxyl, C₁₋₆ alkoxy, halogen or NR⁹R¹⁰;
R³ is hydrogen, halogen, CF₃, OR⁸, COOR⁹, CONR⁹R¹⁰ or SO₂R¹¹;
R⁴ is hydrogen, halogen, CF₃, OR⁹, NR⁹R¹⁰, NR⁹COR¹¹, NR⁹SO₂R¹¹, SO₂NR⁹R¹⁰, or C₁₋₆ alkyl optionally substituted by hydroxyl, C₁₋₆ alkoxy or NR⁹R¹⁰;
or when R³ and R⁴ are positioned ortho and taken together form —O(CH₂)ₙO—, where n is 1-3;
R⁵ is hydrogen or C₁₋₆ alkyl optionally substituted by hydroxyl, C₁₋₆ alkoxy or NR⁹R¹⁰;
R⁶ is hydrogen, C₁₋₆ alkyl, C₁₋₆ alkoxy or NR⁹R¹⁰;
R⁷ is hydrogen, C₁₋₆ alkyl, phenyl or C₁₋₃ alkylphenyl wherein said phenyl groups are optionally substituted by one or more substituents selected from halogen, C₁₋₆ alkyl, CF₃, OCF₃ or OR⁹;
R⁸ is hydrogen or C₁₋₆ alkyl optionally substituted by OR⁹ or NR⁹R¹⁰;
R⁹ is hydrogen, C₁₋₆ alkyl or C₁₋₃ alkylphenyl wherein said phenyl group is optionally substituted by one or more substituents selected from halogen, C₁₋₆ alkyl, CF₃, OR⁸, NR⁹R¹⁰ or OCF₃;
R¹⁰ is hydrogen, C₁₋₆ alkyl, C₁₋₆ alkenyl, phenyl or C₁₋₃ alkylphenyl wherein said phenyl groups are optionally substituted by one or more substituents selected from halogen, C₁₋₆ alkyl, CF₃, OR⁸ or OCF₃;
or the groups R⁹ and R¹⁰ when they are attached to a nitrogen atom may together form a 5- or 6-membered ring which optionally contains one further heteroatom selected from NR⁹, S and O; and
R¹¹ is C₁₋₆ alkyl or a phenyl group optionally substituted by one or more substituents selected from halogen, C₁₋₆ alkyl, CF₃, OCF₃ or OR⁸.

2. A compound as claimed in claim 1 wherein R¹ and R² are independently CHOHCF₃.

3. A compound as claimed in claim 1 wherein
X and Y are independently NR⁵ or O;
W and Z are independently a bond or (CH₂)ₘCH(R⁷)(CH₂)ₙ;
m=0-1 and n=0-2;
R¹ and R² are independently hydrogen, halogen, CF₃, OR⁸, NR⁹R¹⁰, NR⁹COR¹¹, NR⁹SO₂R¹¹ or C₁₋₆ alkyl optionally substituted by hydroxyl, C₁₋₆ alkoxy or NR⁹R¹⁰;
R³ is hydrogen, halogen, CF₃, OR⁸, COOR⁹, CONR⁹R¹⁰ or SO₂R¹¹;
R⁴ is hydrogen, halogen, CF₃, OR⁹, NR⁹R¹⁰, NR⁹COR¹¹, NR⁹SO₂R¹¹ or C₁₋₆ alkyl optionally substituted by hydroxyl, C₁₋₆ alkoxy or NR⁹R¹⁰;
R⁵ is hydrogen or C₁₋₆ alkyl optionally substituted by hydroxyl, C₁₋₆ alkoxy or NR⁹R¹⁰;
R⁶ is hydrogen, C₁₋₆ alkyl, C₁₋₆ alkoxy or NR⁹R¹⁰;
R⁷ is hydrogen, C₁₋₆ alkyl, phenyl or C₁₋₃ alkylphenyl wherein said phenyl groups are optionally substituted by one or more substituents selected from halogen, C₁₋₆ alkyl, CF₃, OCF₃ or OR⁹;
R⁸ is hydrogen or C₁₋₆ alkyl optionally substituted by OR⁹ or NR⁹R¹⁰;
R⁹ is hydrogen, C₁₋₆ alkyl or C₁₋₃ alkylphenyl wherein said phenyl group is optionally substituted by one or more substituents selected from halogen, C₁₋₆ alkyl, CF₃, OR⁸, NR⁹R¹⁰ or OCF₃;
R¹⁰ is hydrogen, C₁₋₆ alkyl, C₁₋₆ alkenyl, phenyl or C₁₋₃ alkylphenyl wherein said phenyl groups are optionally substituted by one or more substituents selected from halogen, C₁₋₆ alkyl, CF₃, OR⁸ or OCF₃;
or the groups R⁹ and R¹⁰ when they are attached to a nitrogen atom may together form a 5- or 6-membered ring which optionally contains one further heteroatom selected from NR⁹, S and O; and
R¹¹ is C₁₋₆ alkyl or a phenyl group optionally substituted by one or more substituents selected from halogen, C₁₋₆ alkyl, CF₃, OCF₃ or OR⁸.

4. A compound as claimed in claim 1, wherein
R¹ and R² are independently hydrogen, halogen, CF₃, OR⁸ or NR⁹R¹⁰;
R³ is hydrogen, F, or OR⁸;
R⁴ is hydrogen, halogen, CF₃, OR⁹ or NR⁹R¹⁰;
R⁵ is hydrogen or C₁₋₆ alkyl optionally substituted by hydroxyl, C₁₋₆ alkoxy or NR⁹R¹⁰;
R⁶ is hydrogen, C₁₋₆ alkyl, C₁₋₆ alkoxy or NR⁹R¹⁰;
R⁷ is hydrogen, C₁₋₆ alkyl;
R⁸ is hydrogen or C₁₋₆ alkyl optionally substituted by NR⁹R¹⁰;
R⁹ is hydrogen, C₁₋₆ alkyl or C₁₋₃ alkylphenyl wherein said phenyl groups are optionally substituted by one or more substituents selected from halogen, C₁₋₆ alkyl, CF₃, OR⁸, NR⁹R¹⁰ or OCF₃;
R¹⁰ is hydrogen, C₁₋₆ alkyl, C₁₋₆ alkenyl, phenyl or C₁₋₃ alkylphenyl wherein said phenyl groups are optionally substituted by one or more substituents selected from halogen, C₁₋₆ alkyl, CF₃, OR⁸ or OCF₃;
or the groups R⁹ and R¹⁰ when they are attached to a nitrogen atom may together form a 5- or 6-membered ring which optionally contains one further heteroatom selected from NR⁹, S and O; and
R¹¹ is C₁₋₆ alkyl or a phenyl group optionally substituted by one or more substituents selected from halogen, C₁₋₆ alkyl, CF₃, OCF₃ or OR⁸.
m=0 and n=0-1

5. A compound selected from
2,5-Bis-(3-hydroxyphenylamino)pyrimidine
2-(3-Hydroxyphenylamino)-5-[3-(trifluoromethyl)phenylamino]pyrimidine
2-(3-Hydroxyphenylamino)-5-[3,4-dichlorophenylamino]pyrimidine
2-(3-Trifluoromethylphenylamino)-5-(3 hydroxyphenylamino)pyrimidine
2-(3-Hydroxyphenylamino)-5-[phenyl(methyl)amino]pyrimidine
2-(3-Hydroxyphenylamino)-5-[3trifluoromethylphenyl(methyl)amino]pyrimidine
2-(3-Hydroxyphenylamino)-5-(4-fluorophenoxy)pyrimidine

6. A pharmaceutical composition comprising a compound as claimed in claim 1, together with one or more pharmaceutically acceptable carriers or excipients.

7. The use of a compound as claimed in claim 1 in the manufacture of a medicament for the treatment of an amyloid-related disease.

8. The use as claimed in claim 7 wherein the medicament is for the treatment of:
a) any form of Alzheimer's disease (AD or FAD);
b) any form of mild cognitive impairment (MCI) or senile dementia;
c) Down's syndrome;
d) cerebral amyloid angiopathy, inclusion body myositis, hereditary cerebral hemorrhage with amyloidosis (HCHWA, Dutch type), or age-related macular degeneration (ARMD);
e) fronto-temporal dementia;
f) any form of Parkinson's disease (PD) or dementia with Lewy bodies;
g) Huntington's disease (HD), dentatorubral pallidoluysian atrophy (DRPLA), spinocerebellar ataxia (SCA, types 1, 2, 3, 6 and 7), spinal and bulbar muscular atrophy (SBMA, Kennedy's disease), or any other polyglutamine disease;
h) Creutzfeldt-Jakob disease (CJD), bovine spongiform encephalopathy (BSE) in cows, scrapie in sheep, kuru, Gerstmann-Straussler-Scheinker disease (GSS), fatal familial insomnia, or any other transmissible encephalopathy that is associated with the aggregation of prion proteins;
i) amyotrophic lateral sclerosis (ALS) or any other form of motor neuron disease;
j) familial British dementia (FBD) or familial Danish dementia (FDD);
k) hereditary cerebral hemorrhage with amyloidosis (HCHWA, Icelandic type);
l) type II diabetes (adult onset diabetes, or non-insulin dependent diabetes mellitus, NIDDM);
m) dialysis-related amyloidosis (DRA) or prostatic amyloid;
n) primary systemic amyloidosis, systemic AL amyloidosis, or nodular AL amyloidosis;
o) myeloma associated amyloidosis;
p) systemic (reactive) AA amyloidosis, secondary systemic amyloidosis, chronic inflammatory disease, or familial Mediterranean fever;
q) senile systemic amyloidosis, familial amyloid polyneuropathy, or familial cardiac amyloid;
r) familial visceral amyloidosis, hereditary non-neuropathic systemic amyloidosis, or any other lysozyme-related amyloidosis;
s) Finnish hereditary systemic amyloidosis;
t) fibrinogen α-chain amyloidosis;
u) insulin-related amyloidosis;
v) medullary carcinoma of the thyroid;
w) isolated atrial amyloidosis;
x) any form of cataract; or
y) any other amyloid-related disease that is associated with the misfolding or aggregation of a specific target amyloid-forming protein or peptide into toxic soluble oligomers, protofibrils, ion channels, insoluble amyloid fibres, plaques or inclusions.

9. A method for the treatment of an amyloid-related disease, which comprises the step of administering to a subject an effective amount of a compound as claimed in claim 1.

10. A method as claimed in claim 9 wherein the amyloid-related disease is any one of those defined in claim 8.

11. An intermediate in the synthesis of a compound of claim 1 of formula (IA) 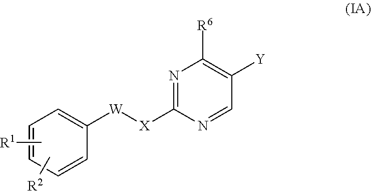
wherein R₁, R₂, W, X and R₆ are as defined in claim 1 and Y is Cl, Br, I or OH.

12. An intermediate as claimed in claim 11 wherein Y is Br.

13. An intermediate in the synthesis of a compound of claim 1 selected from
2-(3-Trifluoromethylphenylamino)-5-bromopyrimidine;
2-(3,4-Dichlorophenylamino)-5-bromopyrimidine;
2-(3-Benzyloxyphenyl-N-tert-butyloxycarbonylamino)-5-bromopyrimidine;
2-(3-Benzyloxyphenyl-N-tert-butyloxycarbonylamino)-5-hydroxypyrimidine;
5-Bromo-2-(N-tert-butyloxycarbonylphenylamino)pyrimidine;
5-Bromo-2-(phenylamino)pyrimidine;
2-(Phenylamino)-5-(4,4,5,5-tetramethyl-[1,3,2]dioxaborolan-2-yl)pyrimidine;
5-Hydroxy-2-(phenylamino)pyrimidine;
1-[4-(5-Bromopyrimidin-2-ylamino)phenyl]-2,2,2-trifluoroethanone; or
1-[4-(5-Bromopyrimidin-2-ylamino)phenyl]-2,2,2-trifluoroethanol.
